(19) United States
(12) Patent Application Publication
(10) Pub. No.: US 2021/0220208 A1
(43) Pub. Date: Jul. 22, 2021

(54) SYSTEM FOR PROVIDING WALKING ENVIRONMENT, SYSTEM FOR SUPPORTING WALKING, AND METHOD FOR PROVIDING WALKING ENVIRONMENT

(71) Applicant: JTEKT CORPORATION, Osaka-shi (JP)

(72) Inventors: Yoshiyuki SHIBATA, Toyota-shi (JP); Manabu KANAYA, Nara-shi (JP); Yuki MATSUOKA, Kariya-shi (JP); Osamu HONDA, Fujiidera-shi (JP); Yusaku YASUI, Nagoya-shi (JP); Yoshinori WADA, Kashiba-shi (JP); Chishin YOHENA, Nara-shi (JP)

(73) Assignee: JTEKT CORPORATION, Osaka-shi (JP)

(21) Appl. No.: 17/153,021

(22) Filed: Jan. 20, 2021

(30) Foreign Application Priority Data
Jan. 22, 2020 (JP) 2020-008734

(51) Int. Cl. A61H 3/00; G08B 21/02; H04W 4/029

(52) U.S. Cl. CPC A61H 3/00; H04L 67/12; H04W 4/029; G08B 21/02

(57) ABSTRACT

There are provided systems for providing a walking environment and supporting walking, and a method for providing a walking environment including: a location information collection step that collects a location information of a walking support equipment; an environmental information collection step that collects an environmental information used upon providing the walking environment while going out; and a process execution step that executes a process of providing the walking environment to an user of the walking support equipment, based on the environmental information collected in the environmental information collection step and the location information collected in the location information collection step.

### CROSS-REFERENCES TO RELATED APPLICATIONS

This application is based on and claims priority under 35 USC 119 from Japanese Patent Application No. 2020-008734 filed on Jan. 22, 2020, the contents of which are incorporated herein by reference.

### TECHNICAL FIELD

The present disclosure relates to a system for providing a walking environment and a system for supporting walking.

### BACKGROUND ART

As shown in JP2019-146755A, a walking support equipment that supports walking of a user is known.

When walking using the walking support equipment, in the case of going out, it is desired to construct a system capable of providing a good walking environment to a user who is out.

### SUMMARY OF INVENTION

The present disclosure provides a system for providing a walking environment and a system for supporting walking, which are capable of providing a good walking environment to a user who is out.

According to an aspect of the present disclosure, a system for providing a walking environment includes: a controller. The controller is configured to execute as various processes: a location information collection step that collects a location information of a walking support equipment that supports walking; an environmental information collection step that collects an environmental information used upon providing the walking environment while going out; and a process execution step that executes a process of providing the walking environment to a user that walks with the walking support equipment, based on the environmental information collected in the environmental information collection step and the location information collected in the location information collection step.

According to the above configuration, in the walking environment, it is possible to provide a walking environment that can improve, for example, a sense of security when using the walking support equipment, the safety when using the walking support equipment, and the efficiency of improving the physical condition by walking using the walking support equipment. Therefore, it is possible to construct a system that provides a good walking environment to a user who is out using the walking support equipment.

If optimization is to be achieved among various methods for realizing the function of the walking environment providing system, the system for providing the walking environment further includes: a network communicator configured to receive the environmental information collected in the environmental information collection step by a communicating via a network.

As the above walking environment, in the case of seeking to improve a sense of security when using the walking support equipment, when the user is wandering, the controller notifies the user or a person concerned with the user of that the user is wandering, by the walking environment provided to the user by the process executed in the process execution step.

According to the above configuration, even if a user who goes out using walking support equipment wanders, the fact is notified to the user or a person concerned with the user, which enables to quickly deal with the wandering of the user. As a result, when going out using the walking support equipment, it is possible to improve a sense of security of the user and a person concerned with the user and to contribute to the construction of a system that provides a good walking environment to the user who is out.

In the above walking environment, in the case of seeking to improve a sense of security when using the walking support equipment, the system for providing the walking environment further includes: a walking environment providing function unit configured to notify the user or a person concerned with the user of that the user is wandering. The environmental information collection step includes collecting an outing area as the environmental information, the outing area being information indicating an area where the user while going out can walk. The process execution step includes an information providing step of providing information indicating that the user is wandering to the walking environment providing function unit when the location information deviates from the outing area.

According to the above configuration, when it is determined that the location information deviates from the outing area, the walking environment providing function unit notifies the user or a person concerned with the user that the pedestrian is wandering, which enables to quickly deal with the wandering of the user. This can contribute to the construction of a system that provides a good walking environment to the user who is out.

In the above walking environment, in the case of seeking to improve the safety when using the walking support equipment, the controller notifies the user of a route suitable for ensuring a walking safety upon using the walking support equipment, by the walking environment provided to the user in the process execution step.

According to the above configuration, the user is notified of a route suitable for ensuring the walking safety, and thus, the walking safety can be improved while the user is out using the walking support equipment. This can contribute to the construction of a system that provides a good walking environment to the user who is out.

In the above walking environment, in the case of seeking to improve the safety when using the walking support equipment, the system for providing the walking environment further includes a walking environment providing function unit configured to notify the user of that the user is heading to a place unsuitable for ensuring the walking safety. The environmental information collection step includes collecting information indicating the place unsuitable for ensuring the walking safety upon using the walking support equipment as the environmental information. The process execution step includes an information providing step of providing information indicating that the user is heading to the place unsuitable for ensuring the walking safety to the walking environment providing function unit when the location information is changed such that the walking support equipment is heading to the place unsuitable for ensuring the walking safety.

According to the above configuration, when the user is heading to a point that is not suitable for ensuring walking safety, the walking environment providing function unit notifies the user of that fact, and thus it is possible to urge the user to go to a place suitable for ensuring the walking safety and to prevent the user from going to a place unsuitable for ensuring the walking safety. As a result, the user can go out using the walking support equipment in an environment suitable for ensuring the walking safety, and thus it is possible to contribute to the construction of a system that provides a good walking environment to the user who is out.

Meanwhile, in the walking environment providing system, it is also possible to construct a system that allows the user to set a destination.

In this case, as the walking environment, in the case of seeking to improve the safety when using the walking support equipment, the system for providing the walking environment further includes a walking environment providing function unit configured to notify the user of a first route among routes to a destination of the user so as to avoid the place unsuitable for ensuring the walking safety upon using the walking support equipment. The controller is configured to further execute a destination information collection step that collects a destination information, the destination information is information indicating the destination of the user. The environmental information collection step includes collecting information indicating the place unsuitable for ensuring the walking safety upon using the walking support equipment as the environmental information. The process execution step includes an information providing step of providing information on the first route to the walking environment providing function unit.

According to the above configuration, the user can be urged to take the first route suitable for ensuring the walking safety using the walking support equipment by notifying the user of the first route that avoids a place that is not suitable for ensuring the walking safety using the walking support equipment by the walking environment providing function unit. As a result, the user can go out using the walking support equipment in an environment suitable for ensuring the walking safety, which can contribute to the construction of a system that provides a good walking environment to the user who is out.

In the system for providing the walking environment, the process execution step includes generating a route information, the route information being information indicating the route suitable for ensuring the walking safety upon using the walking support equipment, and being information configured to notify the user of the route.

According to the above configuration, route information indicating a route suitable for ensuring the walking safety using the walking support equipment is generated and prepared on the system in default. In this case, for example, if there is a request from the user to notify a route suitable for ensuring the walking safety using the walking support equipment, the route information generated and prepared can be used to immediately respond to the user's request.

In the above walking environment, in the case of seeking to improve the efficiency of improving the physical condition by walking using the walking support equipment, the controller notifies the user of a route suitable for improving a physical condition of the user, by the walking environment provided to the user in the process execution step.

According to the above configuration, since the user is notified of the route suitable for improving the physical condition, it is possible to improve the efficiency of improving the physical condition by walking using the walking support equipment. This can contribute to the construction of a system that provides a good walking environment to the user who is out.

In the above walking environment, in the case of seeking to improve the efficiency of improving the physical condition by walking using the walking support equipment, the system for providing the walking environment further includes a walking environment providing function unit configured to notify the user of a second route suitable for increasing efficiency of improving the physical condition of the user, based on a walking ability of the user. The controller is configured to further execute a walking ability information collection step that collects a walking ability information indicating the walking ability of the user. The environmental information collection step includes collecting a load information when the user walks with the walking support equipment at each place as the environmental information. The process execution step includes an information providing step of providing information on the second route to the walking environment providing function unit.

According to the above configuration, the user can be urged to take the second route suitable for improving the physical condition of the user by notifying the user of the second route suitable for improving the efficiency of improving the physical condition of the user by the walking environment providing function unit. As a result, the user can go out using the walking support equipment in an environment where the efficiency of improving the physical condition is improved, which can contribute to the construction of a system that provides a good walking environment to the user who is out.

In the system for providing the walking environment, the walking ability information collection step includes collecting a physiological cost index as the walking ability information, the physiological cost index indicating a load during walking as an index based on a heart rate of the user.

According to the above configuration, in the process execution step, the walking environment providing function unit can notify the user of the second route according to the actual walking ability of the user when the walking support equipment is used.

Meanwhile, if the same route is notified every time, it is conceivable that the user may feel tired of going out using the walking support equipment. In this case, there is a concern that the user's motivation to go out using the walking support equipment will decrease and the efficiency of improving the physical condition will decrease.

On the other hand, as the above-mentioned walking environment, in the case of seeking to improve the efficiency of improving the physical condition by walking using walking support equipment, the system for providing the walking environment further includes a walking environment providing function unit configured to notify the user preferentially of a third route that is different from a history of a walking route while the user is out. The environmental information collection step includes collecting information indicating the history of the walking route of the user while going out as the environmental information. The process execution step includes an information providing step of preferentially providing information on the third route to the walking environment providing function unit.

According to the above configuration, the walking environment providing function unit preferentially notifies the user of the third route that is different from the history of the walking route while the user is out, and thus it is possible to devise ways to make the user feel fresh for going out using the walking support equipment. As a result, the motivation of the user to go out using the walking support equipment can be increased and it is possible to contribute to the construction of a system that provides a good walking environment to the user who is out.

In the system for providing the walking environment, the process execution step includes generating a route information, the route information being information indicating the route suitable for improving the physical condition of the user, and being information configured to notify the user of the route.

According to the above configuration, route information indicating a route suitable for improving the physical condition is generated and prepared on the system in default. In this case, for example, if there is a request from the user to notify a route suitable for improving the physical condition, the route information generated and prepared can be used to immediately respond to the user's request.

According to another aspect of the present disclosure, a system for supporting walking including: a server system including a server controller that executes various processes, a server storage unit that stores various information, and a server network communicator that transmits and receives the various information by communicating via a network; and a walking support equipment including a walking support equipment controller that executes various information and a walking support equipment network communicator that transmits and receives the various information by communicating via the network. The walking support equipment controller builds a system for providing a walking environment configured to execute: a location information collection step that collects a location information of the walking support equipment; an environmental information collection step that collects an environmental information used upon supporting the walking environment while going out by the communicating of the walking support equipment network communicator; and a process execution step that executes a process of providing the walking environment to a user of the walking support equipment, based on the environmental information collected in the environmental information collection step and the location information collected in the location information collection step.

According to the above configuration, as described above, it is possible to construct a walking environment providing system, which is a system capable of providing a good walking environment to a user who is out, by executing a process for providing a walking environment. By realizing the walking environment providing system by the walking support system, it is possible to apply such a walking environment providing system not only to a single walking support equipment but also to a plurality of walking support equipment, which facilitates to expand the business scale. Therefore, it is possible to contribute to the construction of a system in association with a new business model using the walking support equipment.

According to another aspect of the present disclosure, A method for providing a walking environment includes: a location information collection step that collects a location information of a walking support equipment; an environmental information collection step that collects an environmental information used upon providing the walking environment while going out; and a process execution step that executes a process of providing the walking environment to the user, based on the environmental information collected in the environmental information collection step and the location information collected in the location information collection step.

### BRIEF DESCRIPTION OF DRAWINGS

FIG. 1 is a diagram showing a schematic configuration of a walking support system according to a first embodiment;

FIG. 2 is a block diagram showing a schematic configuration of a walking assistant vehicle;

FIG. 3 is a block diagram showing a control configuration of walking support equipment of the first embodiment;

FIG. 4 is a block diagram showing a schematic configuration of a server system according to the first embodiment;

FIG. 5 is a schematic diagram illustrating a walking history and an outing area generated based on the walking history;

FIG. 6 is a sequence chart showing a flow of processing for a user of a walking assistant vehicle to log in to a walking support system and a flow of processing executed when a predetermined operation is input;

FIG. 7 is a flowchart showing a processing flow for the server system to update map data;

FIG. 8 is a flowchart showing a processing flow for providing a walking environment that can improve a sense of security when using walking support equipment;

FIG. 9A is a schematic diagram illustrating the display contents of the display screen of a tablet terminal in the first embodiment;

FIG. 9B is a schematic diagram illustrating the display contents on the display screen of a facility terminal or a family terminal in the first embodiment;

FIG. 9C is a schematic diagram illustrating the display contents of the display screen of a facility terminal or a family terminal in another embodiment;

FIGS. 10A to 10E are schematic diagrams illustrating a method in which a server system generates location status information;

FIG. 11 is a sequence chart showing a flow of processing for a user of a walking assistant vehicle to log in to a walking support system and a flow of processing executed when a predetermined operation is input in a second embodiment;

FIG. 12 is a flowchart showing a flow of processing executed by a tablet terminal in order to provide a walking environment that can improve safety when using walking support equipment;

FIGS. 13A and 13B are schematic diagrams illustrating the display contents of the display screen of the tablet terminal in the second embodiment;

FIG. 14 is a sequence chart showing a flow of processing for a user of a walking assistant vehicle to log in to a walking support system and a flow of processing executed when a predetermined operation is input in a third embodiment;

FIG. 15 is a flowchart showing a flow of processing executed by a tablet terminal in order to provide a walking environment capable of improving the efficiency of improving the physical condition by walking using walking support equipment;

FIG. 16A is a schematic diagram illustrating the display contents of the display screen of the tablet terminal in the third embodiment; and

FIG. 16B is a schematic diagram illustrating the display content of the display screen of the tablet terminal in another embodiment.

### DESCRIPTION OF EMBODIMENTS

### First Embodiment

Hereinafter, a first embodiment in which a walking environment providing system is applied to a walking support system will be described.

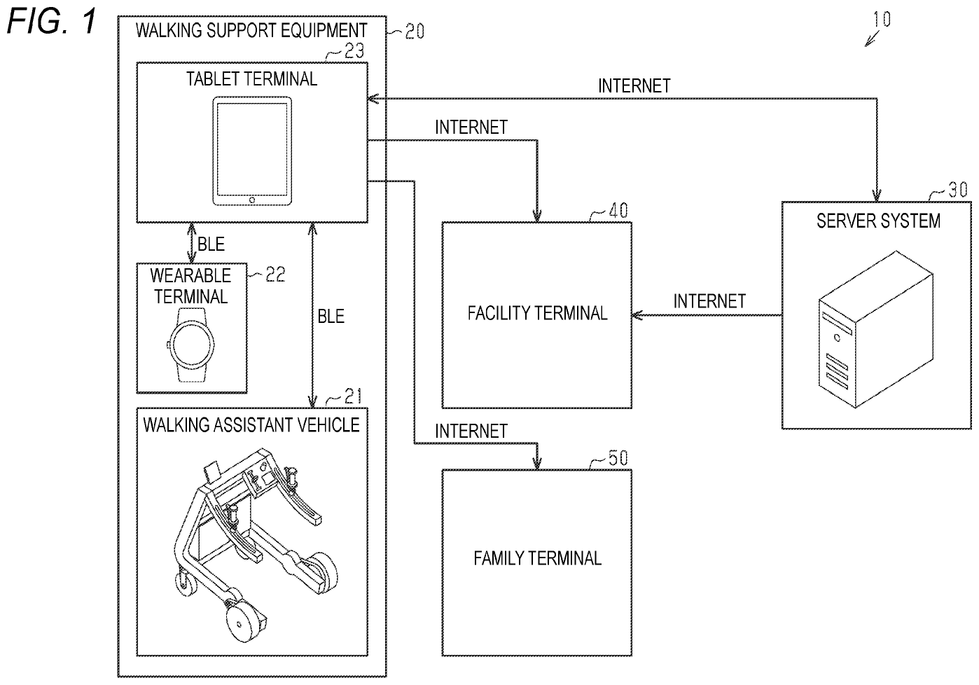

As shown in FIG. 1, a walking support system 10 includes walking support equipment 20, a server system (hereinafter, referred to as “server”) 30, a facility terminal 40, and a family terminal 50, which are connected to each other so as to transmit and receive information via a network such as the Internet. The walking support system 10 is managed and operated by, for example, a management company that manufactures the walking support equipment 20 and rents and manages the walking support equipment 20 for a fee under a rental contract or a lease contract, or a contracted company that outsources the rental management of the walking support equipment 20 from the management company. The walking support system 10 is used to perform a walking movement as an exercise aiming to improve the physical condition of a person to be supported (hereinafter referred to as “user”) such as an elderly person or a person to be cared for who has been certified as requiring long-term care by the Ministry of Health, Labor and Welfare of Japan. In the present embodiment, the improvement of the physical condition means the improvement of the lifestyle, the improvement of the physical ability by the physical function recovery training, and the improvement of the physical ability by the training.

Specifically, the walking support equipment 20 supports the walking movement of a user as an exercise aiming to improve the physical condition by moving on a pedestrian road on which the user walks together with the user who is a pedestrian. The server 30 is realized by a so-called server computer owned by a management company that manufactures and manages the walking support equipment 20, and is a stationary server, a cloud server virtually constructed on a network, or the like. The facility terminal 40 is a computer, a tablet terminal, or the like owned by a support facility used by the user in order to improve the physical condition. The family terminal 50 is a computer, a tablet terminal, or the like owned by the user's family. The pedestrian road is a path on which pedestrians can walk and includes not only sidewalks but also parks and the like. In addition, the pedestrian road may include a place other than a public place such as a private road.

Next, the walking support equipment 20 will be described in detail.

The walking support equipment 20 includes a walking assistant vehicle 21, a wearable terminal 22, and a tablet terminal 23. In the walking support equipment 20, the walking assistant vehicle 21, the wearable terminal 22, and the tablet terminal 23 are paired so as to be basically a set to construct one walking support equipment 20.

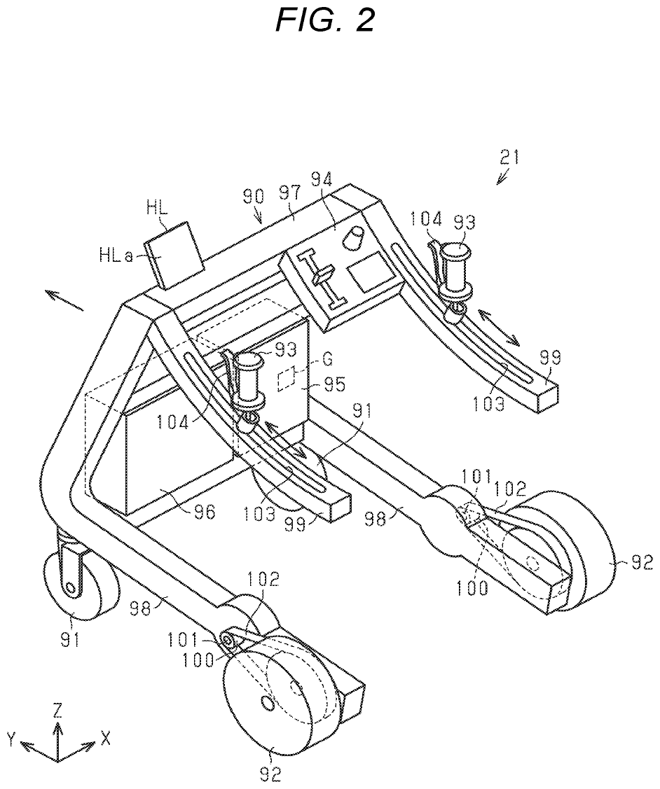

As shown in FIG. 2, the walking assistant vehicle 21 includes a frame 90, a pair of front wheels 91, a pair of rear wheels 92, a pair of handles 93, an operation panel 94, a walking assistant vehicle control unit 95, and a battery 96.

Specifically, the frame 90 has a main body portion 97, a pair of leg portions 98 in the X direction (hereinafter, referred to as “left and right”), and a pair of left and right arm portions 99. The main body portion 97 has a frame shape having a pair of long sides extending to the left and right and a pair of short sides extending in the Z direction (hereinafter, referred to as “up and down”). The pair of leg portions 98 extend in the Y direction (hereinafter referred to as “front and rear”) from the lower ends of the pair of short sides of the main body portion 97 and the pair of arm portions 99 also extend in the front and rear direction from the upper ends of the short sides of the main body portion 97. The pair of leg portions 98 and the pair of arm portions 99 extend in the same direction from the main body portion 97, respectively. In the present embodiment, the walking assistant vehicle 21 is used so that the side on which the pair of leg portions 98 and the pair of arm portions 99 extend from the main body portion 97 is the rear side and the main body portion 97 is the front side.

In the pair of leg portions 98, the front wheels 91 are provided in each front portion so as to form a pair on the left and right and the rear wheels 92 are provided in each rear portion so as to form a pair on the left and right. In the present embodiment, the pair of front wheels 91 are non-driving wheels such as universal casters and the pair of rear wheels 92 are driving wheels such as belt drive.

In the pair of leg portions 98, electric motors 101 having output shafts protruding to the left and right outside of the leg portions 98 are built in the respective portions around the pair of rear wheels 92. Each electric motor 101 is connected to a pulley portion of the rear wheel 92 to which the output shaft corresponds via an endless drive belt 102. The motor torque of each electric motor 101 is transmitted to the corresponding rear wheel 92 via the drive belt 102, so that the pair of rear wheels 92 are belt-driven. In the present embodiment, it is possible to support, that is, assist the walking movement of the user through the forward movement of the walking assistant vehicle 21 accompanying the belt drive of the pair of rear wheels 92.

A rotation sensor 100 such as a rotary encoder is provided on each output shaft of each electric motor 101 provided on each of the pair of leg portions 98. Each rotation sensor 100 detects the rotation number, rotation speed, and the like of the pair of rear wheels 92 per unit time.

The pair of arm portions 99 are provided with handles 93 so as to form a pair on the left and right. The pair of handles 93 are grips that are gripped by the user's left and right hands. The left handle 93, which is one of the pair, is gripped by the left hand of the user who faces the front of the walking assistant vehicle 21 and the right handle 93, which is one of the pair, is gripped by the right hand of the user who faces the front of the walking assistant vehicle 21.

Each of the pair of arm portions 99 has a hollow rod shape and the upper surface thereof is provided with a slit 103 that extends continuously along the longitudinal direction, which is the front-rear direction. Each slit 103 is inserted with one end on the side opposite to the side gripped by the user of the pair of handles 93 so as to be movable in the slit 103. In the present embodiment, the pair of handles 93 move in the front-rear direction along each slit 103 in accordance with the arm swinging motion in the front-rear direction accompanying the walking movement of the user while holding them. As a mechanism that enables the user to swing the arm, each slit 103 may be removed and the pair of handles 93 may be inserted to be slidable in the hollow inner space of the pair of arm portions 99 in the front-rear direction.

Each of the pair of handles 93 is provided with a lever-shaped brake lever 104 that separates forward from the side inserted into each slit 103 with respect to the handle 93 toward the upper side. Each of brake lever 104 has a function of generating a braking force on the corresponding rear wheel 92 through a brake wire (not shown), or reducing the output of the motor torque of the corresponding electric motor 101 when each brake lever 104 is gripped to be close to the corresponding handle 93. In this case, the rotation of the pair of rear wheels 92 is stopped or decelerated.

The operation panel 94 is provided so as to straddle between the right side portion above the long side of the main body portion 97 and the front portion of the right arm portion 99 of the pair. The operation panel 94 is provided with a switch for turning on or off the power of the walking assistant vehicle 21 and a display panel for displaying the on or off state of the power of the walking assistant vehicle 21. The operation panel 94 is a user interface for making the walking assistant vehicle 21 easy for the user to use.

The walking assistant vehicle control unit 95 is provided on the right side portion of the long side below the main body portion 97. The walking assistant vehicle control unit 95 includes, for example, a central processing unit (CPU) and a memory (not shown), and the CPU executes a program stored in the memory to execute various processes on the walking assistant vehicle 21. The walking assistant vehicle control unit 95, as various processes for the walking assistant vehicle 21, executes a process for switching and controlling the power on or off state of the walking assistant vehicle 21, a process for controlling the display content of the operation panel 94, a process for controlling the drive of each electric motor 101, and a process for communicating with the wearable terminal 22 and the tablet terminal 23.

An acceleration sensor G for detecting acceleration, which is a change in speed accompanying the movement of the walking assistant vehicle 21, is provided in a peripheral portion where the walking assistant vehicle control unit 95 is provided. In the present embodiment, the acceleration sensor G is a 3-axis sensor having three detection axes of a “Y-axis” indicating the front-rear direction, an “X-axis” indicating the left-right direction, and a “Z-axis” indicating the up-down direction, so as to correspond to the directions of FIG. 1.

The battery 96 is provided on the left side portion of the long side below the main body portion 97. The battery 96 is, for example, a rechargeable secondary battery and supplies electric power to the operation panel 94, the walking assistant vehicle control unit 95, and each electric motor 101 through an operation of turning on the power of the walking assistant vehicle 21 of the operation panel 94.

A tablet holder HL for holding the tablet terminal 23 is provided on the left side portion above the long side of the main body portion 97 so as to extend upward. The tablet holder HL has a rectangular plate shape having a thickness in the front-rear direction of the walking assistant vehicle 21. The tablet terminal 23 is installed on an installation surface HLa provided on the rear side of the walking assistant vehicle 21, and thus, the tablet terminal 23 is held so as not to be dropped from the walking assistant vehicle 21 in use, such as when moving. By installing the tablet terminal 23 on the tablet holder HL, the user checks the operation and display contents of the tablet terminal 23 without holding the tablet terminal 23 in hand during the use of the walking assistant vehicle 21.

Next, the electrical configuration of the walking assistant vehicle 21 will be described.

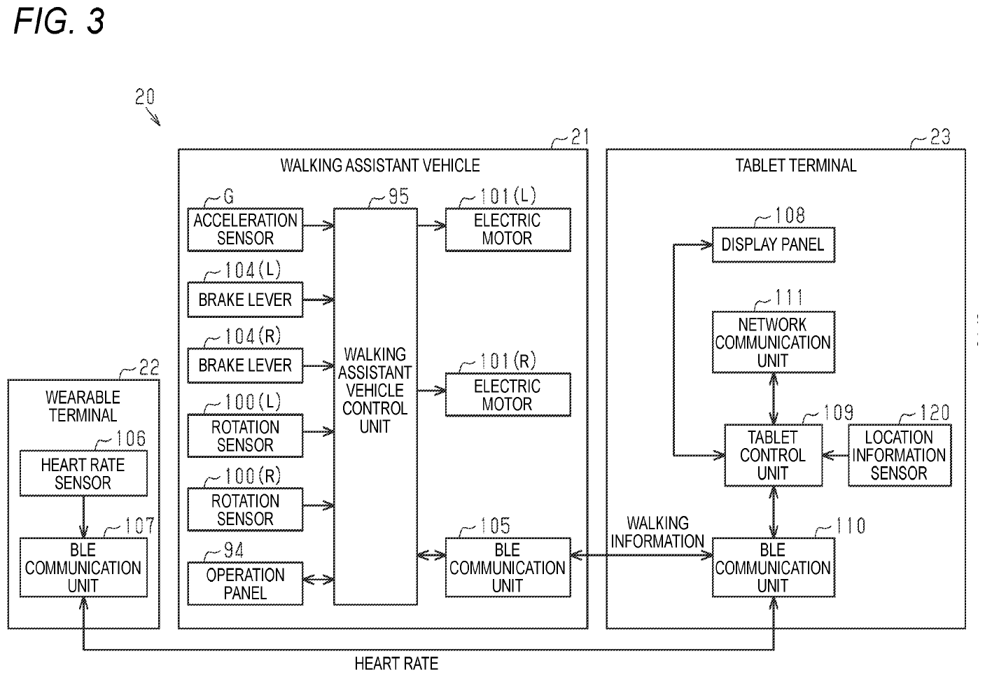

As shown in FIG. 3, in the walking assistant vehicle control unit 95, the operation panel 94, the rotation sensors 100 (left) and 100 (right), and the electric motors 101 (left) and 101 (right), the brake levers 104 (left) and 104 (right) provided corresponding to the respective electric motors 101, a BLE communication unit 105, and the acceleration sensor G are connected to each other. The BLE communication unit 105 realizes the communication function for transmitting and receiving various information to and from the wearable terminal 22 and the tablet terminal 23 through Bluetooth (registered trademark), in particular, BLE (Bluetooth Low Energy) as short-range wireless communication.

The walking assistant vehicle control unit 95 switches and controls the power on or off state of the walking assistant vehicle 21 based on the operation of the operation panel 94, controls the drive of each electric motor 101 by controlling the supply of the current, which is the control amount of each electric motor 101, based on the operation of each brake lever 104, and controls the display contents of the operation panel 94 based on the remaining amount of the battery 96.

Further, based on the detection result of each rotation sensor 100, the walking assistant vehicle control unit 95 calculates walking information regarding the walking speed and the walking distance when the user makes a walking movement using the walking assistant vehicle 21 during one trip, for example, with one trip being during one logged-in state from the allowance to log in to the walking support system 10 described later to the log-out. In the present embodiment, the walking speed is obtained as the maximum speed in one trip. Further, the walking assistant vehicle control unit 95 may calculate the walking speed with a predetermined unit time such as one day (24 hours) as one trip or may calculate the average walking speed as the walking speed.

Then, the walking assistant vehicle control unit 95 transmits the calculated walking information through the BLE communication unit 105 to the tablet terminal 23 that has completed the pairing described later when logging out from the walking support system 10 or when login to the walking support system 10 is allowed during the logged-in state.

Next, the wearable terminal 22 will be described.

The wearable terminal 22 is a wristband type worn by the user on the wrist. The wearable terminal 22 has a communication function for transmitting and receiving various information to and from the walking assistant vehicle 21 and the tablet terminal 23 through, for example, Bluetooth, in particular, BLE, as short-range wireless communication. Further, the wearable terminal 22 may have other functions such as a clock function in addition to the function for using the walking support system 10.

Specifically, as shown in FIG. 3, the wearable terminal 22 includes a heart rate sensor 106. The heart rate sensor 106 detects the heart rate of the user when using the walking assistant vehicle 21 by measuring the blood flow of the wrist of the user wearing the wearable terminal 22.

A BLE communication unit 107 that enables communication by the BLE is connected to the heart rate sensor 106. Then, the heart rate detected by the heart rate sensor 106 is transmitted through the BLE communication unit 105 to the tablet terminal 23 that has completed the pairing described later while logged in to the walking support system 10.

Next, the tablet terminal 23 will be described.

As short-range wireless communication, the tablet terminal 23 has a communication function for transmitting and receiving various information to and from the walking assistant vehicle 21 and the wearable terminal 22 through, for example, Bluetooth, in particular, the BLE, and a communication function for transmitting and receiving various information to and from the server 30 through the network. Further, the tablet terminal 23 is a general-purpose or dedicated tablet in which a dedicated application for using the walking support system 10 is installed and other applications such as games may be installed.

Specifically, as shown in FIG. 3, the tablet terminal 23 includes a display panel 108 that is a display screen of the tablet terminal 23. The display panel 108 is a touch panel that displays various menu screens for starting an application for using the walking support system 10, and logging in to and logging out from the walking support system 10, can be operated by the user directly touching the various menu screens with a finger and detects a position touched by the user's finger, for example, in an electrostatic method, a pressure sensitive method, or an optical method. In addition, the display panel 108 displays the information on the walking time, walking speed, and walking distance of the walking movement using the walking assistant vehicle 21, and the heart rate before, after, and during the walking movement.

A tablet control unit 109 is connected to the display panel 108. The tablet control unit 109 includes, for example, a central processing unit (CPU) and a memory (not shown), and the CPU executes a program stored in the memory to execute various processes on the tablet terminal 23. As various processes for the tablet terminal 23, the tablet control unit 109 executes a process for detecting the position where the user touches the display panel 108 with a finger, a process for controlling the display contents of the display panel 108, a process for communicating with the walking assistant vehicle 21 and the wearable terminal 22, and a process for communicating with the server 30.

The tablet control unit 109 is connected to a BLE communication unit 110 that enables communication by the BLE and a network communication unit 111 that enables communication by the network. The tablet control unit 109 acquires the walking information calculated by the walking assistant vehicle 21, which has completed the pairing described later, and transmitted through the BLE communication unit 105, and collects the transmitted walking information through the BLE communication unit 110, for example, at the time of logging in to the walking support system 10 or during the logged-in state. Further, the tablet control unit 109 acquires the heart rate detected by the wearable terminal 22, which has completed the pairing described later, and transmitted through the BLE communication unit 107, and collects the transmitted heart rate through the BLE communication unit 110, for example, at the time of logging in to the walking support system 10 or during the logged-in state. Further, the tablet control unit 109 transmits a pairing request, which is information for requesting pairing for constructing the walking support equipment 20, to the walking assistant vehicle 21 and the wearable terminal 22 through the BLE communication unit 107.

Then, the tablet control unit 109 calculates the information on the walking time, walking speed, and walking distance of the walking movement using the walking assistant vehicle 21, and the heart rate before, after, and during the walking movement, and usage information which is the location information of the walking support equipment 20, based on the walking information and the heart rate collected from the walking assistant vehicle 21 and the wearable terminal 22. In the present embodiment, the usage information is the information collected and obtained while the user is out using the walking support equipment 20 and is the information obtained from the walking support equipment 20 as a result of using the walking support equipment 20, that is, the walking assistant vehicle 21 by the user.

Further, a location information sensor 120 that detects location information is connected to the tablet control unit 109. The location information sensor 120 is a GPS sensor that receives, for example, a positioning signal from an artificial satellite for the global positioning system (GPS) and detects the current location (latitude and longitude) and orientation of the tablet terminal 23 based on the received positioning signal. The location information sensor 120 generates the location information of the tablet terminal from the location and orientation of the tablet terminal 23. For example, the tablet control unit 109 acquires the location information generated by the location information sensor 120 at the time of logging in to the walking support system 10 or during the logged-in state and collects the acquired location information. In the present embodiment, the location information indicates the current location of the walking assistant vehicle 21 used in pairing with the tablet terminal 23 and indicates the location of the user who is using the walking assistant vehicle 21 as an alternative.

Further, the tablet control unit 109 transmits the walking information and heart rates collected from the walking assistant vehicle 21 and the wearable terminal 22, and the usage information calculated based on the walking information and the heart rates together with the login information described later, to the server 30 through the network communication unit 111, for example, at the time of logging in to the walking support system 10 or during the logged-in state. Further, the tablet control unit 109 transmits login information, which is the information for requesting the server 30 for login allowance described later for the walking support system 10, to the server 30 through the network communication unit 111. Further, the tablet control unit 109 transmits the wandering information described later to the facility terminal 40 and the family terminal 50 through the network communication unit 111.

The server 30 has a communication function for transmitting and receiving various information between the tablet terminal 23 of the walking support equipment 20 and the facility terminal 40 through the network.

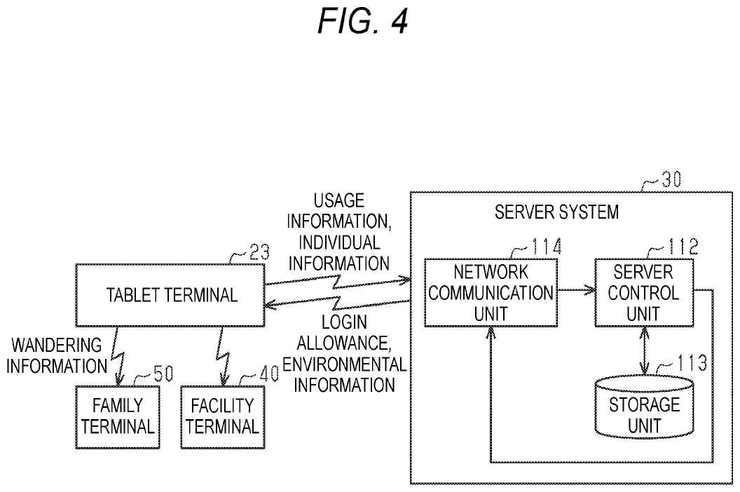

Specifically, as shown in FIG. 4, the server 30 includes a server control unit 112. The server control unit 112 includes, for example, a CPU and a memory (both not shown) and the CPU executes a program stored in the memory to execute various processes on the tablet terminal 23. The various processes executed by the server control unit 112 for managing and operating the walking support system 10 will be described in detail later.

The server control unit 112 is connected to a storage unit 113 as a server storage unit and a network communication unit 114 as a server network communication unit that enables communication via the network. The server control unit 112 acquires login information of the tablet terminal 23 through the network communication unit 114, for example, in response to a login request from the tablet terminal 23 and stores the acquired login information in a predetermined storage area of the storage unit 113 in which RAM, ROM, or the like is adopted. Further, the server control unit 112 records, in a predetermined storage area of the storage unit 113 through the network communication unit 114, the usage information collected through the network communication unit 111 of the tablet terminal 23 that has been allowed to log in, for example, during the login allowance described later or during the logged-in state of the tablet terminal 23.

The storage unit 113 connected to the server control unit 112 stores and manages the map data constructed by associating map information with each location on the ground as information used when providing a walking environment, which is an environment when the walking support equipment 20 is used. The map data is information that is a basis and a material for providing a usage environment, that is, a walking environment for a user who is out. As such a walking environment, in the present embodiment, a walking environment that can improve a sense of security when using the walking support equipment 20 is provided.

Specifically, the storage unit 113 allocates a dedicated storage area for each location on the ground (hereinafter, referred to as “location individual area”), and stores and manages map information regarding the location on the ground to which the location individual area is allocated. That is, the storage unit 113 stores map data constructed by associating map information with each location on the ground. The map information includes basic information created by a map provider, such as address, building name, building shape, road name, road shape, signal location, and the like regarding the location on the ground, and location status information obtained through the network so as to be added to the basic information. In the present embodiment, the location status information is information including information indicating the road surface condition, for example, unevenness, slopes, the height difference, the presence or absence of a groove, and the like, which changes the movement of the walking support equipment 20 when the user walks using the walking support equipment 20 at the location, the traffic volume of automobiles, bicycles, and passersby around the location, and the weather around the location, with respect to the location indicated by the location information. In addition, the location status information is information including, for example, the width of the sidewalk, the load of walking when passing through the location, the presence or absence of a signal or a pedestrian crossing around the location, and the walking history of the user, with respect to the location indicated by the location information.

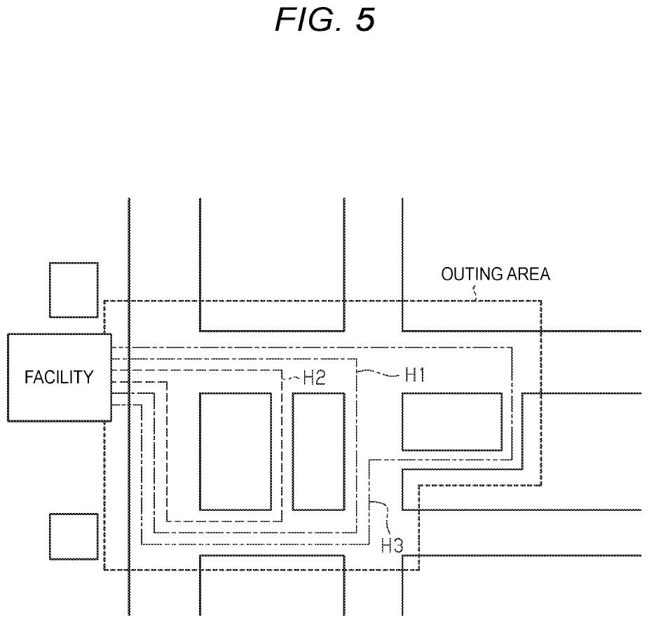

As an example, FIG. 5 shows an outing area (indicated by a short broken line in the drawing), which is an area where a user who is out can walk. The outing area is an area formed by collecting location information when it is assumed that a user who is out using the walking support equipment 20 normally walks, and an area when it is assumed that the user walks so as to deviate from this area or the user is wandering. In addition, wandering may be caused by a decrease in the user's memory or judgment, or may be caused by the user not knowing where he/she is or his/her destination. Further, the deviation means that any of the location information constituting the outing area does not match the current location information of the user and occurs when the user moves from the inside to the outside of the outing area. Such an outing area is set to include a walking history based on the walking history (indicated by “H1 to H3” in the drawing) configured by collecting the location information so as to be a locus of the location information that changes as the user walks. The walking history is a movement history when the walking support equipment 20 moves on the pedestrian road. The generation of the outing area, which is the environmental information of the present embodiment, will be described later.

As shown in FIG. 4, when the user goes out using the walking support equipment 20, upon receiving an outing request transmitted from the tablet terminal 23 in response to a predetermined operation performed by the user on the tablet terminal 23, the server control unit 112 transmits the outing area generated based on the walking history of the user recorded in the individual area described later to the tablet terminal 23. Further, the server control unit 112 collects the information that is the basis of a walking history of the user, that is, the location information detected by the information sensor 120, while the user is out using the walking support equipment 20 while logged in to the walking support system 10. The server control unit 112 generates a walking history of the user based on the location information and updates the map data. The walking history collected during this logged-in state is used when generating an outing area to be used during the next and subsequent logged-in states.

Hereinafter, various processes executed by the server control unit 112 for managing and operating the walking support system 10 will be described in detail.

First, a description will be made for a process executed by the walking assistant vehicle 21, the wearable terminal 22, the tablet terminal 23, and the server 30 when the user logs in to the walking support system 10. In the following, a description will be made for the flow of processing for allowing login to the walking support system 10 by the walking assistant vehicle 21, the wearable terminal 22, the tablet terminal 23, and the server 30 by executing periodic processing for each control cycle.

In the present embodiment, the user or a person concerned with the user needs a predetermined registration act in the walking support system 10 in order to use the walking support system 10. In addition, the person concerned with the user includes family members including relatives of the user, as well as persons who can represent the registration act of the user, such as the staff of the support facility being used.

The storage unit 113 connected to the server control unit 112 allocates a dedicated storage area (hereinafter referred to as “individual area”), and records and manages various information regarding the user who is the target of the registration act in the individual area, together with login information which is registration information dedicated to each user by the user's registration act. At the time of the prescribed registration act, it is required to provide individual information including the contact information of the user such as the name, address, and e-mail address, the contact information of a person concerned with the user such as the name, address, and e-mail address, and the facility name of the support facility used by the user. The individual information is recorded and managed in the individual area allocated by the user's registration act together with the login information, which is the registration name and the password for security set for each user upon completion of the predetermined registration act.

After the completion of the predetermined registration act, the user at the walking support system 10, that is, in the support facility using the walking support equipment 20 starts the application for using the walking support system 10 through the operation of the display panel 108 of the tablet terminal 23 of the support facility.

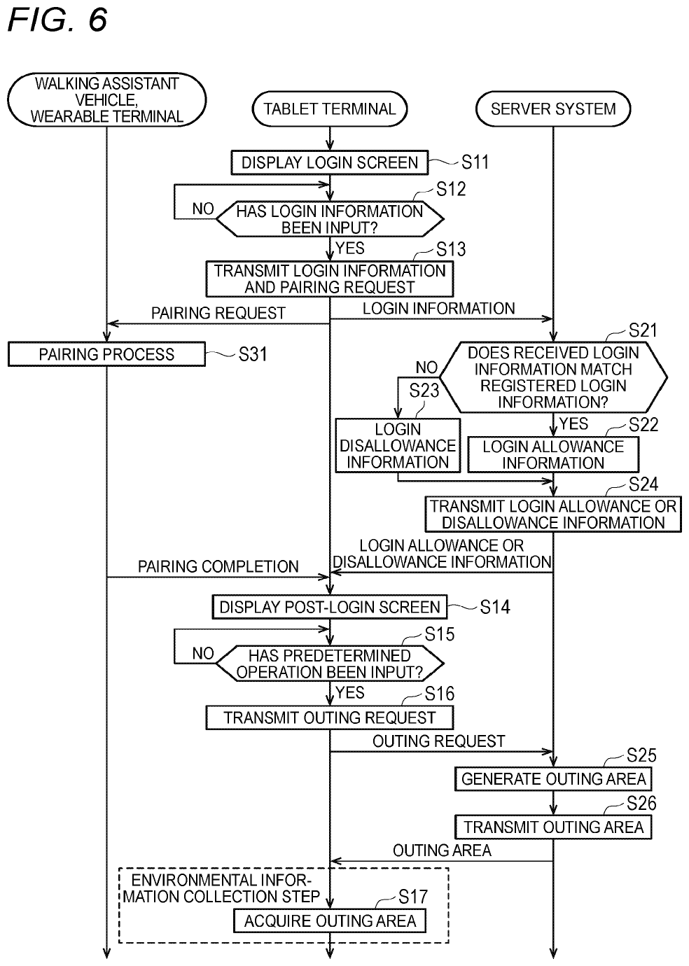

In this case, as shown in FIG. 6, in the tablet terminal 23, the tablet control unit 109 displays a login screen that is the menu screen for logging in to the walking support system 10 with the starting of the application for using the walking support system 10 (step S11). Subsequently, the tablet control unit 109 determines whether or not the login information has been input (step S12) and repeatedly executes the process of step S12 until the login information is input (step S12: NO).

When it is determined that the login information has been input (step S12: YES), the tablet control unit 109 transmits the login information input in step S12 to the server 30 and transmits a pairing request for constructing the walking support equipment 20 to the walking assistant vehicle 21 and the wearable terminal 22 (step S13). In this case, the tablet control unit 109 transmits the individual identification information that can identify the individual of the tablet terminal 23 to the server 30. Since the tablet terminal 23 is paired with a predetermined walking assistant vehicle 21 or wearable terminal 22 in principle, the individual identification information can be used to identify the individual walking assistant vehicle 21 or wearable terminal 22 on the server 30.

In the walking assistant vehicle 21, upon receiving the pairing request transmitted by the tablet terminal 23, the walking assistant vehicle control unit 95 executes the pairing process for authenticating each other so that the corresponding tablet terminal 23 is used as a communication partner and another tablet terminal 23 existing at a short distance is not used as a communication partner (step S31). In step S31, when the wearable terminal 22 receives the pairing request transmitted by the tablet terminal 23, like the walking assistant vehicle control unit 95, the wearable terminal 22 executes the pairing process for authenticating each other so that the corresponding tablet terminal 23 is used as a communication partner and another tablet terminal 23 existing at a short distance is not used as a communication partner. When the authentication and the pairing with the tablet terminal 23 that has transmitted the pairing request is completed, the walking assistant vehicle control unit 95 and the wearable terminal 22 transmit information indicating the pairing completion to the tablet terminal 23 that has transmitted the ring request.

On the other hand, in the server 30, upon receiving the login information transmitted by the tablet control unit 109, the server control unit 112 determines whether or not the login information matches the registered login information (step S21). If the login information received in step S21 matches the registered login information (step S21: YES), the server control unit 112 generates login allowance information indicating that the login to the walking support system 10 is allowed (Step S22). In this case, the server control unit 112 completes the allowance of the user's login to the walking support system 10 and transmits the generated login allowance information to the tablet terminal 23 so that various information about the user can be recorded or updated in the individual area of the storage unit 113 (step S24).

On the other hand, if the login information received in step S21 does not match the registered login information (step S21: NO), the server control unit 112 generates login disallowance information indicating that the login to the walking support system 10 is not allowed (step S23). In this case, the server control unit 112 transmits the generated login disallowance information to the tablet terminal 23 without completing the allowance of the user's login to the walking support system 10 (step S24).

In the tablet terminal 23, when the tablet control unit 109 receives the pairing completion transmitted by the walking assistant vehicle 21 and the wearable terminal 22 to which the pairing request is transmitted, and receives the login allowance information transmitted by the server 30, the tablet terminal 109 causes the display panel 108 to display a post-login screen indicating the allowance to log in to the walking support system 10 (step S14). If the tablet control unit 109 does not receive the pairing completion transmitted by the walking assistant vehicle 21 and the wearable terminal 22 to which the pairing request is transmitted, or if the tablet control unit 109 receives the login disallowance information transmitted by the server 30, the tablet terminal 109 causes the display panel 108 to display an error screen that allows the user to understand that the allowance has not been granted.

Next, the process executed by the server 30 to generate the map data that is the basis for providing a walking environment will be described. In the present embodiment, the server 30 updates the walking history of the user among the map data. The server control unit 112 of the server 30 executes a process for updating the map data that is the basis for providing a walking environment for each control cycle.

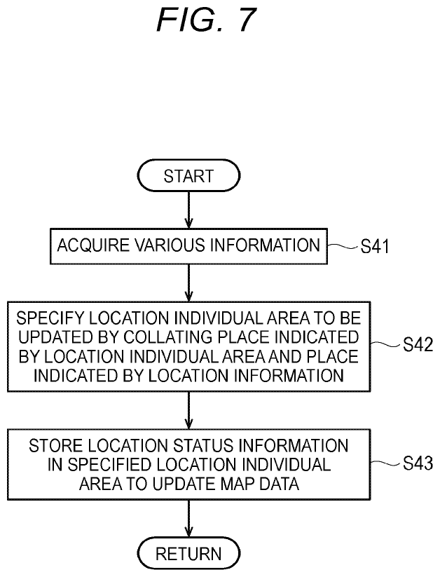

As shown in FIG. 7, the server control unit 112 acquires various information through the network (step S41). In the present embodiment, the various information acquired in step S41 includes the usage information collected by the walking support equipment 20 through the network and the information acquired from other than the walking support equipment 20 through the network.

The usage information acquired in step S41 includes information on the detection result of the acceleration sensor G in a state of being associated with the location information detected by the location information sensor 120 of the tablet terminal 23. In addition, the usage information acquired in step S41 also includes information on the detection result of the heart rate detected by the heart rate sensor 106 in a state of being associated with the location information detected by the location information sensor 120 of the tablet terminal 23. Further, the various information acquired in step S41 includes the location information detected by the location information sensor 120 of the tablet terminal 23. The server control unit 112 can generate a walking history from the locus of the location information configured by collecting the location information collected during the current logged-in state. The walking history is information that is the basis for generating an outing area and the outing area is information that is the basis for providing a walking environment that can improve a sense of security when using the walking support equipment 20. The server control unit 112 generates location status information by evaluating the pedestrian road at the location based on the information indicating the condition of the pedestrian road at the location indicated by the location information. In the present embodiment, the location status information is device acquisition information obtained from the walking support equipment 20 as a result of using the walking support equipment 20.

The information obtained through the network from other than the walking support equipment 20 includes information indicating the traffic volume of automobiles, bicycles, and passersby, the weather, grooves, sidewalk widths, the presence or absence of obstacles, and the like. The information obtained through the network from other than the walking support equipment 20 is associated with the location information indicating the corresponding location. The information from other than the walking support equipment 20 is obtained from, for example, a posting-type Internet service such as a social networking service, an Internet service provided by a road management company, an Internet service provided by a weather forecast company, and the like. The server control unit 112 can generate location status information by evaluating the pedestrian road at the location based on various information about the location indicated by such location information. Such location status information is information obtained from a network other than the walking support equipment 20.

The server control unit 112 collates the location indicated by the location individual area with the location indicated by the location information detected by the location information sensor 120 in step S41 with respect to the location individual area of the storage unit 113, and specifies the location individual area to be updated (step S42).

The server control unit 112 stores the location status information associated with the location information corresponding to the location to which the location individual area is allocated in the location individual area to be updated which has been specified in step S42, thereby updating the map data (step S43). The map data updated in this way is the information that is the basis for providing a walking environment. In the present embodiment, since the map data is updated including the information obtained from the walking support equipment 20 as a result of using the walking support equipment 20, the map data can be said as map data for the pedestrian road in which information evaluated about the pedestrian road on which the walking support equipment 20 moves is collected. The map data is information that can be displayed with map information about each location scattered when visually displayed on each terminal. The map data also includes information that is not used to provide a walking environment that can improve a sense of security when using the walking support equipment 20. When generating an outing area that is the basis for providing a walking environment that can improve a sense of security when using the walking support equipment 20, information on the walking history of the user is extracted from the map data and used as information necessary for the walking environment to be provided. An outing area is generated as environmental information based on the walking history of the user extracted from the map data.

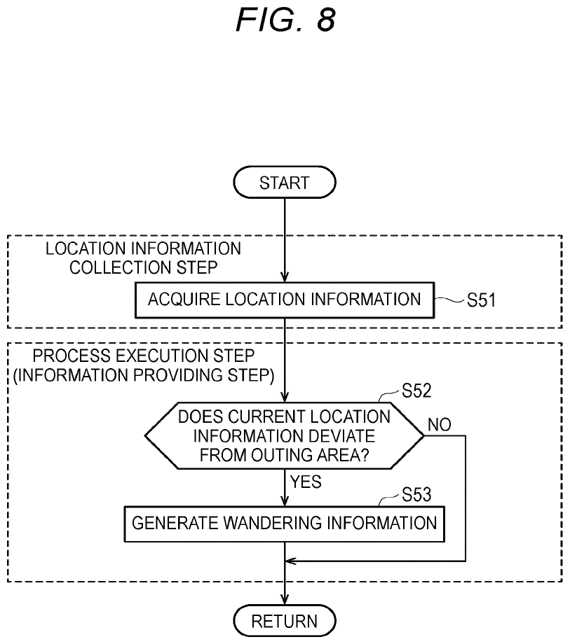

Next, a description will be made for the process executed by the tablet terminal 23 and the server 30 when the user goes out using the walking support equipment 20 after the completion of the login allowance and before the execution of the process shown in FIG. 8 described later, which is a process executed by the user while going out using the walking support equipment 20.

As shown in FIG. 6, the tablet control unit 109 determines whether or not a predetermined operation to be performed on the tablet terminal 23 has been input when the user goes out using the walking support equipment 20 (step S15), and the process of step S15 is repeatedly executed until the predetermined operation is input (step S15: NO).

If the tablet control unit 109 determines that the predetermined operation to be performed on the tablet terminal 23 has been input when the user goes out using the walking support equipment 20 (step S15: YES), the tablet control unit 109 transmits an outing request to the server 30 (step S16).

In the server 30, upon receiving the outing request transmitted by the tablet control unit 109, as a step of generating environmental information, the server control unit 112 generates an outing area based on the walking history of the user stored in the location individual area (step S25) and transmits the outing area to the tablet terminal 23 (step S26). The server 30 extracts, from the map data, the walking history of the user necessary to provide a usage environment that can improve a sense of security when using the walking support equipment 20 and generates the outing area based on the walking history. In step S25, the server control unit 112 generates an outing area to be used during the current logged-in state based on the walking history collected during the past logged-in state so as to include all the walking histories H1 to H3 shown in FIG. 5, for example. Specifically, the server control unit 112 generates, as an outing area, an area obtained by connecting areas within a circle having a predetermined radius centered on the individual location information for a plurality of location information constituting the walking history. The predetermined radius is set to a distance at which the user who goes out using the walking support equipment 20 is normally walking and does not deviate. In the present embodiment, the outing area can be generated by various methods, for example, the area of the pedestrian road only in the walking history.

Further, the tablet control unit 109 transmits the above-mentioned outing request to the server 30 (step S16), and then acquires the outing area transmitted from the server control unit 112 as an environmental information collection step of collecting environmental information (step S17). In this way, the tablet terminal 23 acquires the outing area when going out using the walking support equipment 20 after the login allowance is completed. In the present embodiment, the process of step S17 executed by the tablet control unit 109 corresponds to the environmental information collection step.

Next, the process executed by the tablet terminal 23 with respect to the location information collected and obtained while the user is out using the walking support equipment 20 will be described. The following process is executed for each control cycle by the tablet control unit 109 of the tablet terminal 23. The following process is executed after the tablet terminal 23 acquires the outing area by transmitting the outing request to the server 30.

As shown in FIG. 8, the tablet control unit 109 acquires the location information detected by the location information sensor 120 (step S51). In the present embodiment, the process of step S51 executed by the tablet control unit 109 corresponds to the location information collection step.

The tablet control unit 109 determines whether or not the location information acquired in step S51 deviates from the outing area acquired by transmitting the outing request to the server 30 (step S52). In step S52, the tablet control unit 109 determines whether or not the location information constituting the outing area matches the location information acquired in step S51. In the present embodiment, the process of step S52 executed by the tablet control unit 109 and the process of step S53 below correspond to the information providing step as a process execution step.

If the location information acquired in step S51 does not deviate from the outing area acquired by transmitting the outing request to the server 30 (step S52: NO), that is, if the location information constituting the corresponding outing area matches the location information acquired in step S51, the tablet control unit 109 determines that the user is not wandering and terminates the process.

If the location information acquired in step S51 deviates from the outing area acquired by transmitting the outing request to the server 30 (step S52: YES), that is, if the location information constituting the corresponding outing area does not match the location information acquired in step S51, the tablet control unit 109 determines that the user is wandering. In this case, the tablet control unit 109 generates wandering information, which is information for notifying that the user is wandering (step S53). When the wandering information is generated in step S53, the tablet control unit 109 transmits the wandering information to the facility terminal 40 and the family terminal 50. In the present embodiment, the tablet control unit 109 that executes the location information collection step, the environmental information collection step, and the process execution step through the processes of steps S17 and S51 to S53 corresponds to a walking support equipment control unit. Further, in the present embodiment, the network communication unit 111 corresponds to the network communication unit for walking support equipment.

Hereinafter, a description will be made for the display contents displayed on the tablet terminal 23, the facility terminal 40, and the family terminal 50 while the user is out using the walking support equipment 20.

When wandering information is generated while the user is out using the walking support equipment 20, the tablet control unit 109 controls a display content of the display panel 108 so as to notify that the user is wandering.

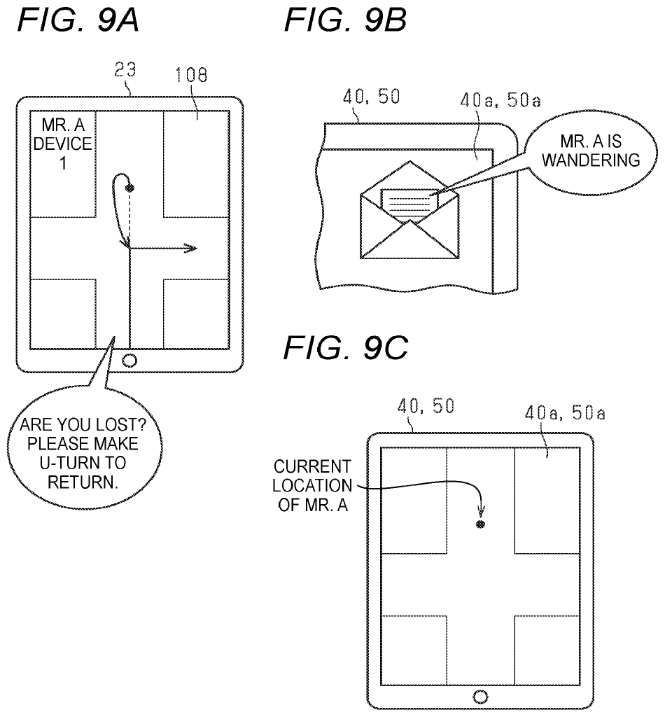

In this case, as shown in FIG. 9A, the display panel 108 of the tablet terminal 23 displays a display content including an image identifying the user (“Mr./Ms. A”) and the individual walking assistant vehicle 21 (“Device 1”), an image identifying a map around the current location of the walking support equipment 20 and an image for urging the user to return to the outing area. In addition, as such an image urging to return to the outing area, for example, a message image such as “Are you lost? Please make a U-turn to return” is displayed on the display panel 108 of the tablet terminal 23. In the present embodiment, the display panel 108 of the tablet terminal 23 that notifies by displaying an image for urging to return to the outing area corresponds to a walking environment providing function unit. In the present embodiment, the tablet control unit 109 generates the wandering information so as to be able to be used in the process for notifying with the display panel 108 of the tablet terminal 23, thereby providing the wandering information to the display panel 108.

In addition, when the wandering information is generated, the tablet terminal 23 identifies the contact information of the user's family or the contact information of the support facility used by the user, which is included in the individual information through the server 30, and transmits the wandering information to the contacts by using the e-mail addresses of the registered facility terminal 40 or family terminal 50.

In this case, as shown in FIG. 9B, when the facility terminal 40 or the family terminal 50 receives the wandering information, an image of an e-mail notifying the wandering of the user (“Mr./Ms. A is wandering”) is displayed on the display panel 40a that is the display screen of the facility terminal 40 or on the display panel 50a that is the display screen of the family terminal 50. In the present embodiment, the display panel 40a of the facility terminal 40 or the display panel 50a of the family terminal 50 for notifying the wandering of the user through image display corresponds to the walking environment providing function unit.

The operation of the present embodiment will be described.

According to the present embodiment, in order to improve a sense of security when using the walking support equipment 20 when the user goes out using the walking support equipment 20, the tablet control unit 109 in the tablet terminal 23 executes the process of step S52 of determining whether the location information detected by the location information sensor 120 deviates from the outing area acquiring by transmitting the outing request to the server 30. Further, the tablet control unit 109 can notify the user and a person concerned with the user of the wandering of the user by using the result of the determination in step S52. Therefore, in the present embodiment, even if the user who goes out using the walking support equipment 20 wanders, that fact is notified to the user or a person concerned with the user, and thus a quick response can be made for the wandering of the user.

The effects of the present embodiment will be described.

(1) According to the present embodiment, it is possible to improve a sense of security of the user and a person concerned with the user when the user goes out using the walking support equipment 20 and it is possible to contribute to the construction of a system that provides a good walking environment to the user who is out.

(2) According to the present embodiment, as described in the above-mentioned effect (1), it is possible to construct a walking environment providing system, which is a system capable of providing a good walking environment to the user who is out, by executing the processing for providing the walking environment. By realizing the walking environment providing system by the walking support equipment 20, the walking support equipment 20 can add not only a function of supporting the walking of the user but also a good walking environment for the user who is out. As a result, the commercial value of the walking support equipment 20 is increased, which can lead to an increase in the distribution amount of the walking support equipment 20 to the market and facilitates to expand the business scale using the walking support equipment 20.

### Second Embodiment

Next, a second embodiment in which the walking environment providing system is applied to the walking support system will be described. The same configurations as those of the above-described embodiment are designated by the same reference numerals and the duplicated description will be omitted.

In the present embodiment, as the walking environment, a walking environment that can improve the safety when using the walking support equipment 20 is provided. Specifically, it provides a walking environment that can prevent the user from going to a place that is not suitable for ensuring the walking safety (hereinafter referred to as “caution place”) while going out using the walking support equipment 20. In the present embodiment, the environmental information is a caution place map that is information for grasping the caution places.

As shown in FIG. 4, in the server 30, when the server control unit 112 receives the outing request transmitted by the tablet control unit 109, the server control unit 112 transmits a caution place map as environmental information to the tablet terminal 23.

The map data generated by the process executed by the server 30 to generate the map data that is the basis for providing a walking environment also includes information that is not used to provide a walking environment that can improve the safety when using the walking support equipment 20. When generating a caution place map that is the basis for providing a walking environment that can improve the safety when using the walking support equipment 20, for example, information indicating that there are a rough road, a stepped road, and the like will be extracted from the map data and used as information necessary for the walking environment to be provided. Based on the information extracted from the map data, a caution place map is generated as environmental information.

Next, the process executed by the server 30 to generate the location status information will be described. The server control unit 112 of the server 30 executes the process for generating location status information for each control cycle. Here, as an example of the generation of the location status information, the generation of the location status information based on the detection result of the acceleration sensor G will be described. From the detection result of the acceleration sensor G, for example, information such as that the walking support equipment 20 has moved on a rough road, has moved on an inclined road, has moved on a stepped road, has collided with a step, or has fallen can be calculated. The server control unit 112 generates location status information based on this information.

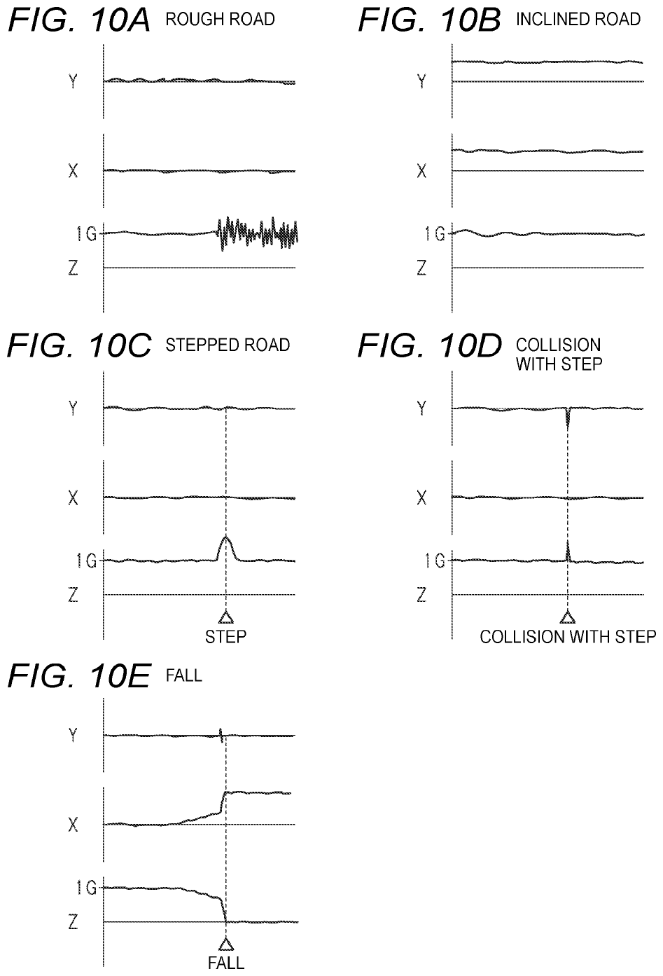

FIG. 10A shows the detection result of the acceleration sensor G when the walking support equipment 20 passes through a rough road, which is a pedestrian road having unevenness that makes repeated vertical vibration. In this case, as a result of repeating the vertical vibration of the walking support equipment 20, the vibration appears as a continuous vertical change in the “Z-axis” and appears as only a steady change in both the “X-axis” and the “Y-axis”. The server control unit 112 can determine that the location indicated by the associated location information is a rough road based on, for example, the detection result of the acceleration sensor G appearing as a continuous vertical change of the “Z-axis”. The server control unit 112 can estimate the size of the unevenness of the rough road based on the size of the continuous vertical change in the “Z-axis”. For example, in the case of large unevenness where the walking support equipment 20 is likely to fall, information indicating that a fall is likely to occur is generated as the location status information for the location indicated by the location information. The location status information is information indicating a caution place extracted at the time of generating a caution place map as information necessary for providing a usage environment that can improve the safety during use.

FIG. 10B shows the detection result of the acceleration sensor G when the walking support equipment 20 passes through an inclined road that is a pedestrian road having a slope in either front, back, left, or right. In this case, as a result of the walking support equipment 20 moving in a tilted state, the detection result of the acceleration sensor G shows that the “Y-axis” and the “X-axis” show a steady tilt. The server control unit 112 can determine that the location indicated by the associated location information is an inclined road based on, for example, the detection result of the acceleration sensor G showing a steady inclination in the “Y-axis” and the “X-axis”. The server control unit 112 can estimate the magnitude of the slope of the inclined road based on the magnitude of the steady inclination in the “Y-axis” and the “X-axis” in the detection result of the acceleration sensor G. For example, in the case of a large inclined road where the walking support equipment 20 is likely to fall, information indicating that a fall is likely to occur is generated as the location status information for the location indicated by the location information. The location status information is information indicating a caution place extracted at the time of generating a caution place map as information necessary for providing a usage environment that can improve the safety during use.

FIG. 10C shows the detection result of the acceleration sensor G when the walking support equipment 20 passes through a stepped road, which is a pedestrian road having a step with a large height difference. In this case, as a result of the walking support equipment 20 moving so as to cross the step, the detection result of the acceleration sensor G appears as a change having one large peak of the “Z-axis” and appears as only a steady change in both the “Y-axis” and the “X-axis”. The server control unit 112 can determine that the location indicated by the associated location information is a stepped road based on, for example, the detection result of the acceleration sensor G appearing as a change having one large peak of the “Z-axis” and appearing as only a steady change in both the “Y-axis” and the “X-axis”. The server control unit 112 can also estimate the size of the step based on the size of the peak of the “Z-axis” in the detection result of the acceleration sensor G. For example, when there is a large step where the walking support equipment 20 is likely to fall, information indicating that a fall is likely to occur is generated as the location status information for the location indicated by the location information. The location status information is information indicating a caution place extracted at the time of generating a caution place map as information necessary for providing a usage environment that can improve the safety during use.

FIG. 10D shows the detection result of the acceleration sensor G when the walking support equipment 20 collides with a step. In this case, as a result of the walking support equipment 20 colliding with the step and generating a large impact in the front-rear and up-down directions, the impact appears as a change having one large peak of the “Y-axis” and the “Z-axis” and appears as only a steady change in the “X-axis”. The server control unit 112 can determine that a collision with the step has occurred at the location indicated by the associated location information based on, for example, the detection result of the acceleration sensor G appearing as a change having one large peak of the “Y-axis” and the “Z-axis” and appearing as only a steady change in the “X-axis”. For example, when the walking support equipment 20 has frequently collided with the step in the past, the server control unit 112 generates information indicating that a collision with the step is likely to occur as the location status information for the location indicated by the location information. A collision with a step is a precursor that may lead to a fall of the walking support equipment 20. Therefore, the location status information is information indicating a caution place extracted at the time of generating a caution place map as information necessary for providing a usage environment that can improve the safety during use.

FIG. 10E shows the detection result of the acceleration sensor G when the walking support equipment 20 falls. In this case, as a result of the walking support equipment 20 falling and generating a small impact in the front-rear direction and a large impact in the left-right direction, the impact appears as a relatively small and short change in the “Y-axis” and appears as a change having one large peak in the “X-axis”. In this case, by further rolling over, the “X-axis” that is originally detected with the left and right acceleration of the acceleration sensor G is detected with the vertical acceleration, and the “Z-axis” that is originally detected with the vertical acceleration is detected with the left and right acceleration. This effect is reflected so that if the acceleration by a predetermined gravity (“1G” in the drawing) is detected in the “X-axis”, the acceleration by the predetermined gravity is not detected in the “Z-axis”. The server control unit 112 can determine that the walking support equipment 20 has fallen at the location indicated by the associated location information, based on, for example, the detection result of the acceleration sensor G appearing as a relatively small and short change in the “Y-axis” and appearing as a change having one large peak in the “X-axis”. For example, when the walking support equipment 20 has frequently fallen in the past, the server control unit 112 generates information indicating that a fall is likely to occur as the location status information for the location indicated by the location information. The location status information is information indicating a caution place extracted at the time of generating a caution place map as information necessary for providing a usage environment that can improve the safety during use.

Next, as an example of the generation of the location status information, the generation of the location status information based on the traffic volume around the pedestrian road acquired through the network will be described. When there is a lot of traffic around the location, the server control unit 112 generates information indicating that there is a lot of traffic and caution is required when walking, as the location status information for the location indicated by the location information. The location status information is information indicating a caution place extracted at the time of generating a caution place map as information necessary for providing a usage environment that can improve the safety during use.

Next, the processes executed by the tablet terminal 23 and the server 30 will be described.

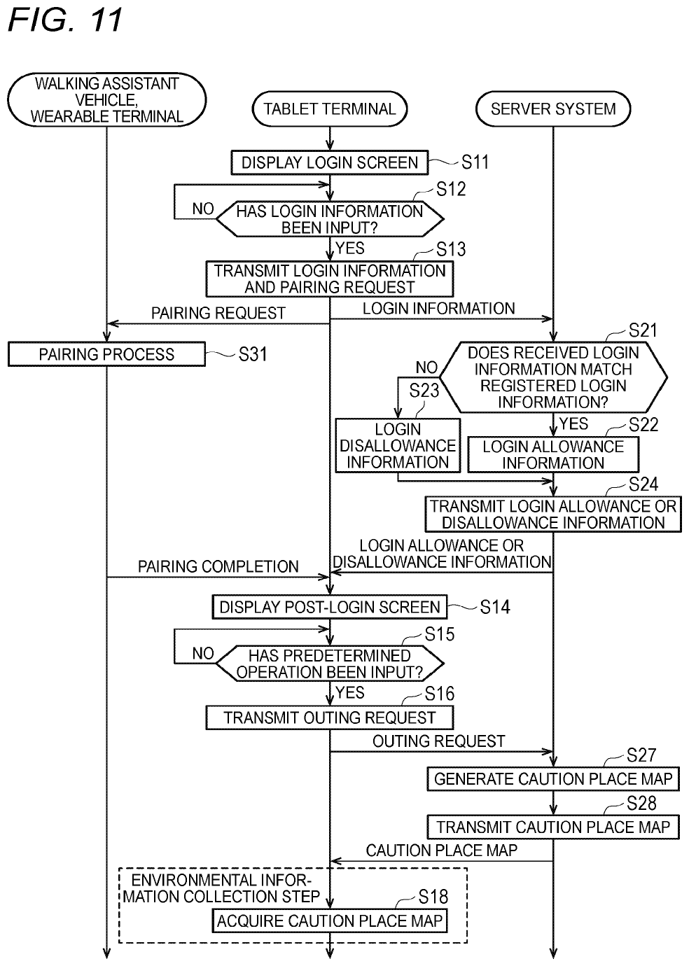

As shown in FIG. 11, as a step of generating environmental information, upon receiving the outing request transmitted by the tablet control unit 109, the server control unit 112 generates a caution place map, which is information showing so that caution places are grasped (step S27) and transmits the caution place map to the tablet terminal 23 (step S28). When a caution place map is generated, the server 30 extracts, from the map data, the information necessary for providing a usage environment that can improve the safety during use when the walking support equipment 20 is used, and generates a caution place map based on the extracted information in step S27. The information to be extracted includes the road surface condition, such as unevenness, slopes, the presence or absence of a groove, and the like, which changes the movement of the walking support equipment 20 when the user walks using the walking support equipment 20 at the location, and information such as traffic volume near the location. The caution place map generated in step S27 is configured by collecting caution places around the support facility having the walking support equipment 20 that has been requested to go out. The server control unit 112 specifies the location of the support facility having the walking support equipment 20 that has been requested to go out based on the login information. A caution place is, for example, a place on the pedestrian road or its surroundings where there are unevenness, slopes, or steps that make walking difficult, there is heavy traffic enough to hinder walking, or the weather is not suitable for walking.

After transmitting the above-mentioned outing request to the server 30 (step S16), the tablet control unit 109 acquires the caution place map transmitted from the server control unit 112 as an environmental information collection step of collecting environmental information (step S18). In this way, the tablet terminal 23 acquires the caution place map when going out using the walking support equipment 20 after the login allowance is completed.

Next, the process executed by the tablet terminal 23 with respect to the location information collected and obtained while the user is out using the walking support equipment 20 will be described. The following process is executed for each control cycle by the tablet control unit 109 of the tablet terminal 23. The following process is executed after the tablet terminal 23 has acquired the caution place map by transmitting the outing request to the server 30.

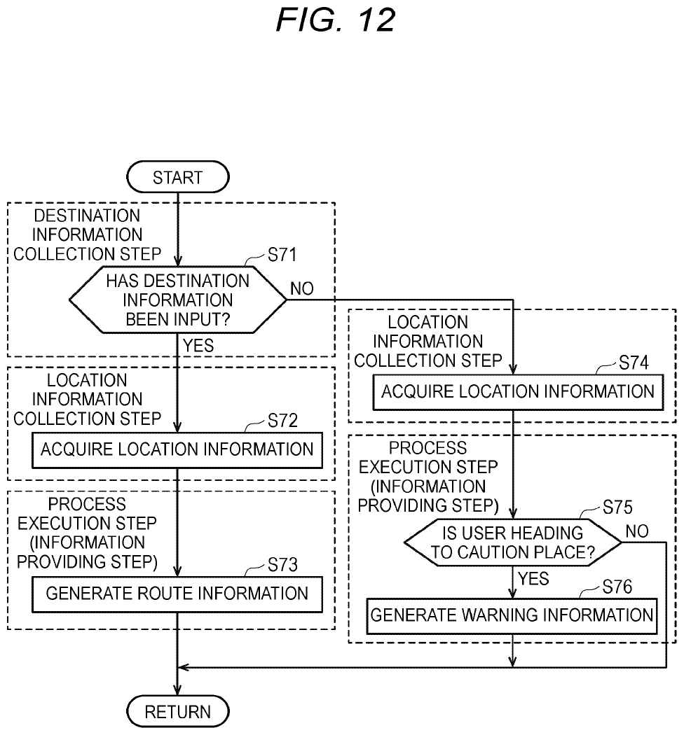

As shown in FIG. 12, as a destination information collection step of collecting destination information, which is information indicating the user's destination, the tablet control unit 109 determines whether or not the destination information, which is the information indicating the destination, has been input in order to set the destination of the user who is out this time (step S71).

As a location information collection step for collecting location information, if the destination information is input (step S71: YES), the tablet control unit 109 acquires the location information detected by the location information sensor 120 (step S72).

Subsequently, as an information providing step of providing information on a first route to the display panel 108, which is a walking environment providing function unit having a function of notifying the first route that avoids the caution places among the routes from the current location to the destination, the tablet control unit 109 generates route information indicating the first route based on the caution place map acquired through the server 30 (step S73). In step S73, the tablet control unit 109 extracts a route so as to avoid the caution places shown in the caution place map with reference to the caution place map, as a route connecting the destination indicated in the destination information acquired in step S71 and the current location indicated in the location information acquired in step S72. In step S73, the tablet control unit 109 extracts, for example, the shortest route so as to be the shortest regardless of the caution place map, as a route connecting the destination indicated in the destination information input in step S71 and the current location indicated in the location information acquired in step S72, and generates route information indicating the shortest route as route information indicating a route different from the first route.

On the other hand, as a location information collection step of collecting location information, if the destination information is not input (step S71: NO), the tablet control unit 109 acquires the location information detected by the location information sensor 120 (step S74).

Next, as an information providing step of providing warning information, which is information indicating that the user is heading to a caution place to the walking environment providing function unit having a function of notifying that the user is heading to a caution place, the tablet control unit 109 determines whether or not the user is heading to a caution place based on the caution place map acquired through the server 30 (step S75). In step S75, the tablet control unit 109 determines whether or not the current location indicated in the location information acquired in step S74 is close to a caution place.

The tablet control unit 109 ends the process if the user is not heading to a caution place (step S75: NO), that is, if the user's current location is far from a caution place and is not close to a caution place. If the user is heading to a caution place (step S75: YES), that is, if the user's current location is close to a caution place, the tablet control unit 109 generates warning information, which is information indicating that the user is heading to a caution place (step S76).

Hereinafter, the display contents displayed on the tablet terminal 23 while the user is out using the walking support equipment 20 will be described.

When the user is out using the walking support equipment 20 and the route information is generated by the process of step S73, as a request from the user for notifying the first route, provided that there is an operation by the user for the request, the tablet control unit 109 controls the display contents of the display panel 108 so as to be a display content notifying the first route based on the route information indicating the first route among the route information generated in the process of step S73. On the other hand, as a request from the user for notifying the first route, provided that there is no operation by the user for the request and, for example, there is an operation by the user for a request from the user for notifying the shortest route, the shortest route is notified based on the route information indicating the shortest route among the route information generated in the process of step S73. In this case, the tablet control unit 109 may execute the processes of step S75 and step S76 together.

That is, if the first route is notified as a route notification, the route information indicating the shortest route among the route information generated in step S73 is not used in the function of notifying the user of the route although it is generated. On the other hand, if the shortest route is notified as a route notification, the route information indicating the first route among the route information generated in step S73 is not used in the function of notifying the user of the route although it is generated. The specifications of the walking support system 10 may be such that the first route is notified as a default when the destination information is set.

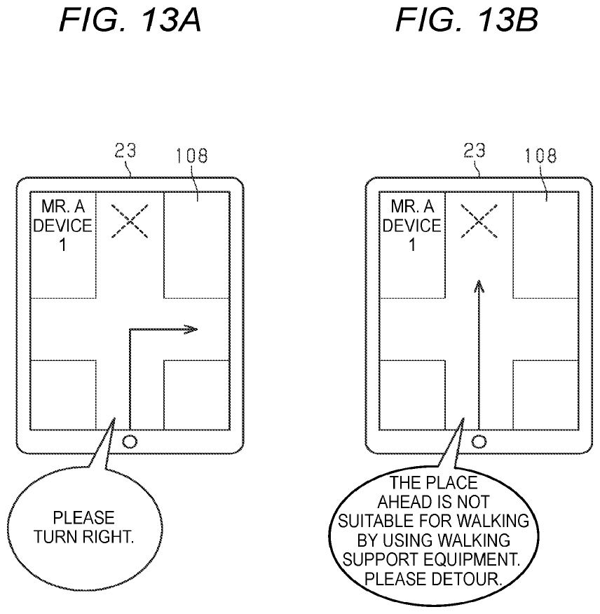

In this case, as shown in FIG. 13A, the display panel 108 of the tablet terminal 23 displays a display content including an image identifying the user (“Mr./Ms. A”) and the individual walking assistant vehicle 21 (“Device 1”), an image for identifying the first route, and an image for urging the user to take the first route. As such an image for urging the user to take the first route, for example, a message image such as “Please turn right” is displayed on the display panel 108 of the tablet terminal 23. In the present embodiment, the display panel 108 of the tablet terminal 23 that notifies the user by displaying an image for urging the user to take the first route corresponds to the walking environment providing function unit. In the present embodiment, the tablet control unit 109 generates the route information so as to be able to be used in the process for notifying with the display panel 108 of the tablet terminal 23, thereby providing the route information to the display panel 108.

Further, when the warning information is generated while the user is out using the walking support equipment 20, the tablet control unit 109 controls the display contents of the display panel 108 so as to notify that the user is heading to a caution place.

In this case, as shown in FIG. 13B, the display panel 108 of the tablet terminal 23 displays a display content including an image for identifying the user (“Mr./Ms. A”) and the individual walking assistant vehicle 21 (“Device 1”), an image for identifying the caution place, and an image for urging the user not to go to the caution place. In addition, as such an image for urging the user not to go to the caution place, for example, a message image such as “The place ahead is not suitable for walking using walking support equipment. Please detour.” is displayed on the display panel 108 of the tablet terminal 23. In the present embodiment, the display panel 108 of the tablet terminal 23 that notifies by displaying an image for urging the user not to go to the caution place corresponds to the walking environment providing function unit. In the present embodiment, the tablet control unit 109 generates warning information so as to be able to be used in the process for notifying with the display panel 108 of the tablet terminal 23, thereby providing the warning information to the display panel 108.

The operation of this embodiment will be described.

According to the present embodiment, when the user goes out using the walking support equipment 20, in order to improve the safety when using the walking support equipment 20, the tablet control unit 109 in the tablet terminal 23 executes the process of step S73 and the processes of steps S75 and S76. In the process of step S73, among the routes from the current location to the destination, the first route that avoids the caution places is notified. As a result, the user can be urged to take the first route. Further, in the process of step S76, when the user is heading to a caution place, the user is notified that he/she is heading to the caution place. As a result, when the user is heading to a caution place, that fact is notified to the user by the display panel 108 of the tablet terminal 23, which can urge the user to go to a place suitable for ensuring the walking safety and prevent the user from going to the caution place.

The effects of the present embodiment will be described.

(3) According to the present embodiment, the user can go out using the walking support equipment 20 in an environment suitable for ensuring the walking safety, which can contribute to the construction of a system that provides a good walking environment to the user who is out.

(4) According to the present embodiment, route information indicating the first route suitable for ensuring the walking safety using the walking support equipment 20 is generated and prepared on the system in default. In this case, if there is a request from the user for notifying the route information indicating a route suitable for ensuring the walking safety using the walking support equipment 20, the route information generated and prepared can be used to immediately respond to the user's request.

### Third Embodiment

Next, a third embodiment in which the walking environment providing system is applied to the walking support system will be described. The same configurations as those of the above-described embodiments are designated by the same reference numerals and the duplicated description will be omitted.

In the present embodiment, as the walking environment, a walking environment that can improve the efficiency of improving the physical condition by walking using the walking support equipment 20 is provided. Specifically, a walking environment is provided in which a place where the user can give a load to the user through walking while going out using the walking support equipment 20 (hereinafter, referred to as a “load place”) is incorporated into the route. In the present embodiment, the environmental information is a load place map that is information showing so that the load place can be grasped. The load place is set based on the location status information, which is the information indicating the road surface condition, for example, unevenness, slopes, the presence or absence of a groove, and the like, which changes the movement of the walking support equipment 20 when the user walks using the walking support equipment 20 at the location indicated by the location information.

The map data generated by the process executed by the server 30 to generate the map data that is the basis for providing the walking environment also includes information that is not used to provide a walking environment that can improve the efficiency of improving the physical condition by walking using the walking support equipment 20. When generating a load place map that is the basis for providing a walking environment that can improve the efficiency of improving the physical condition by walking using the walking support equipment 20, information indicating an inclined road, a stepped road, or the like is extracted from the map data and used as information necessary for the walking environment to be provided. A load place map is generated as environmental information based on the information extracted from the map data.

Next, the process executed by the server 30 to generate the location status information will be described. The server control unit 112 of the server 30 executes a process for generating location status information for each control cycle. Here, as an example of the generation of the location status information, the generation of the location status information based on the detection result of the acceleration sensor G will be described.

As shown in FIG. 10B, the server control unit 112 estimates the magnitude of the slope of the inclined road based on the magnitude of the steady inclination of the “Y-axis” and “X-axis” in the detection result of the acceleration sensor G. For example, when there is an inclined road, information indicating that there is a walking load is generated as the location status information for the location indicated by the location information. The location status information is information indicating the load places extracted at the time of generating the load place map as information necessary for providing a walking environment that can improve the efficiency of improving the physical condition by walking using the walking support equipment 20.

As shown in FIG. 10C, the server control unit 112 estimates the size of the step based on the size of the peak of the “Z-axis” in the detection result of the acceleration sensor G. For example, when there is a step, information indicating that there is a walking load is generated as the location status information for the location indicated by the location information. The location status information is information indicating the load places extracted at the time of generating the load place map as information necessary for providing a walking environment that can improve the efficiency of improving the physical condition by walking using the walking support equipment 20.

Next, as an example of the generation of the location status information, the generation of the location status information based on the detection result of the heart rate sensor 106 will be described.

When the increase in heart rate at the location is large, the server control unit 112 generates information indicating that the increase in heart rate is large and there is a walking load as the location status information for the location indicated by the location information. The location status information is information indicating the caution places extracted at the time of generating a caution place map as information necessary for providing a walking environment that can improve the safety during use. The location status information is information indicating the load places extracted at the time of generating the load place map as information necessary for providing a walking environment that can improve the efficiency of improving the physical condition by walking using the walking support equipment 20.

As shown in FIG. 4, in the server 30, upon receiving the outing request transmitted by the tablet control unit 109, the server control unit 112 transmits the load place map and the walking history as environmental information to the tablet terminal 23. Further, the server control unit 112 reads various information of the usage information stored in the storage unit 113 and based on the various information, calculates the physiological cost index (PCI), which is a physiological cost index indicating the load during the walking movement as an index, and then, updates the contents of the corresponding individual areas of the storage unit 113. PCI is calculated as a [beat/m] dimensional value using a commonly used calculation such as [post-walking heart rate (beat/minute)−resting heart rate (beat/minute)]/walking speed (m/minute). In the present embodiment, the heart rate after the walking movement from the usage information is used as the “post-walking heart rate” and the heart rate before the walking movement from the usage information is used as the “resting heart rate”. The individual area of the storage unit 113 stores the PCI calculated as the history of the PCI, for example, after the user's registration act is performed. In the server 30, upon receiving the outing request transmitted by the tablet control unit 109, the server control unit 112 transmits the user's PCI recorded in the individual area of the storage unit 113 to the tablet terminal 23.

In the present embodiment, as a step of collecting the basic information for providing a walking environment, the server control unit 112 collects the location status information acquired by replacing the caution place in the second embodiment with the load place. This location status information is collected by the walking support equipment 20 and some is collected through the network other than the walking support equipment 20, as in the second embodiment. Further, the server control unit 112 collects the walking history in the same manner as in the first embodiment in order to collect the information that is the basis for providing the walking environment.

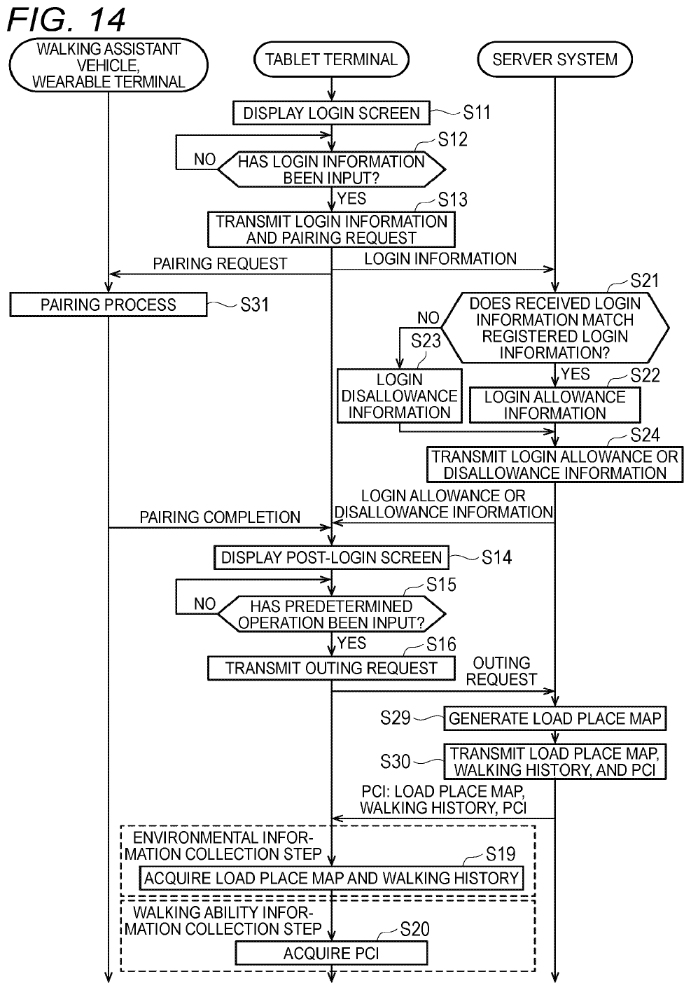

As shown in FIG. 14, as a step of generating environmental information, the server control unit 112 generates a load place map upon receiving the outing request transmitted by the tablet control unit 109 (step S29) and transmits the load place map, the walking history, and the user's PCI to the tablet terminal 23 (step S30). When generating the load place map to be generated in step S27, information necessary for providing a walking environment that can improve the efficiency of improving the physical condition by walking using the walking support equipment 20 is extracted from the map data to generate a load place map based on the extracted information. The information to be extracted includes information such as the road surface condition, such as unevenness or slopes, which changes the movement of the walking support equipment 20 when the user walks using the walking support equipment 20 at the location, and heart rate. The load place map generated in step S29 is configured by collecting load places around the support facility having the walking support equipment 20 that has been requested to go out. The server control unit 112 specifies the location of the support facility having the walking support equipment 20 that has been requested to go out based on the login information. The load place is, for example, a place on the pedestrian road or its surroundings where there are unevenness, slopes, or steps that can give a load to the user through walking.

After transmitting the above-mentioned outing request to the server 30 (step S16), the tablet control unit 109 acquires the load place map and the walking history transmitted from the server control unit 112 as an environmental information collection step of collecting environmental information (step S19), and acquires the PCI of the user as a walking ability information collection step of collecting the walking ability information indicating the walking ability of the user (step S20). In this way, the tablet terminal 23 acquires the load place map, the walking history, and the user's PCI when going out using the walking support equipment 20 after the login allowance is completed.

Next, the process executed by the tablet terminal 23 with respect to the location information collected and obtained while the user is out using the walking support equipment 20 will be described. The following process is executed for each control cycle by the tablet control unit 109 of the tablet terminal 23. The following process is executed after the tablet terminal 23 acquires the load place map, the walking history, and the user's PCI by transmitting the outing request to the server 30.

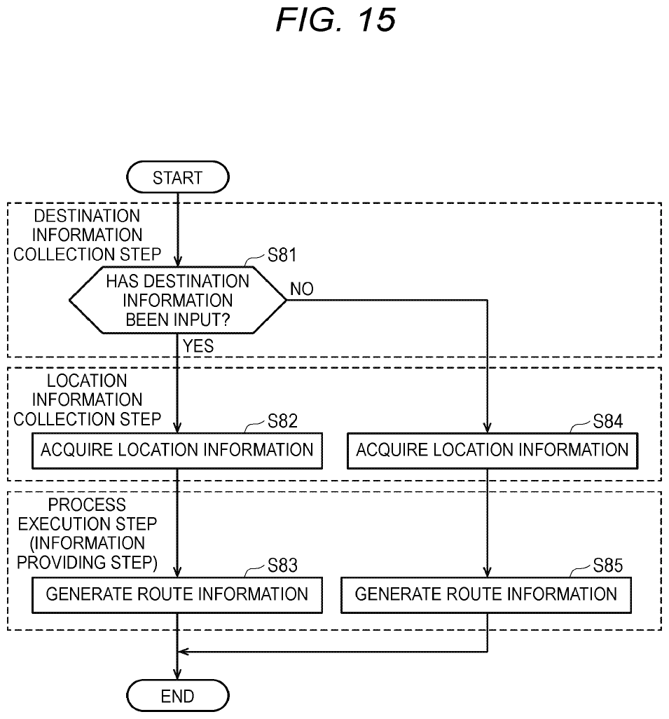

As shown in FIG. 15, as a destination information collection step of collecting destination information, which is information indicating the user's destination, the tablet control unit 109 determines whether or not the destination information, which is information indicating the user's destination while going out this time, has been input (step S81).

As a location information collection step of collecting location information, if the destination information is input (step S81: YES), the tablet control unit 109 acquires the location information detected by the location information sensor 120 (step S82).

As an information providing step of providing the route information of the optimal route, which is a route that can give a load to the user through walking and can motivate the user to go out using the walking support equipment 20 among the routes from the current location to the destination, the tablet control unit 109 executes the following process. Specifically, the tablet control unit 109 generates route information indicating the optimal route based on the load place map and walking history acquired through the server 30 (step S83).

In step S83, the tablet control unit 109 extracts a second route as the optimal route for improving the user's PCI with reference to the load place map among the routes connecting the destination indicated in the destination information input in step S81 and the current location indicated in the location information acquired in step S82. In this case, for example, for a user, the higher the PCI, the more load is applied to extract a route that improves the PCI, or for a user with a high PCI but a low physical ability in the first place, a route that does not force a load is extracted because simply walking leads to improvement in PCI. When extracting the optimal route in this way, the tablet control unit 109 preferentially extracts a third route from the second routes as the optimal route that makes the user feel fresh when going out with reference to the walking history. In step S83, the tablet control unit 109 extracts the shortest route so as to be the shortest, regardless of the load place map and the walking history, as a route connecting the destination indicated in the destination information input in step S81 and the current location indicated in the location information acquired in step S82, and route information indicating the shortest route is generated as route information indicating a route different from the optimal route.

On the other hand, as a location information collection step of collecting location information, if the destination information is not input (step S81: NO), the tablet control unit 109 acquires the location information detected by the location information sensor 120 (step S84).

Subsequently, as an information providing step, the tablet control unit 109 generates route information indicating the optimal route based on the load place map and the walking history acquired through the server 30 (step S85). In step S85, the tablet control unit 109 extracts a route among the routes starting from the current location indicated in the location information acquired in step S84 and returning to the current location in the same manner as in step S83.

Hereinafter, the display contents displayed on the tablet terminal 23 while the user is out using the walking support equipment 20 will be described.

If the route information is generated by the processes of steps S83 and S85 while the user is out using the walking support equipment 20, as a request from the user for notifying the user of the optimal route, provided that there is an operation by the user for the request, the tablet control unit 109 controls the display contents of the display panel 108 so as to be a display content notifying the user of the optimal route, based on the route information indicating the optimal route among the route information generated in the processes of steps S83 and S85. On the other hand, as a request from the user for notifying the optimal route, provided that there is no operation by the user for the request and there is an operation by the user for a request from the user for notifying the shortest route, for example, the shortest route is notified based on the route information indicating the shortest route among the route information generated in the processes of steps S83 and S85.

That is, if the optimal route is notified as a route notification, the route information indicating the shortest route among the route information generated in steps S83 and S85 is not used in the function of notifying the user of the route although it is generated. On the other hand, when the shortest route is notified as a route notification, the route information indicating the optimal route among the route information generated in step S73 is not used in the function of notifying the user of the route although it is generated. The specifications of the walking support system 10 may be such that the optimal route is notified as a default when the destination information is set.

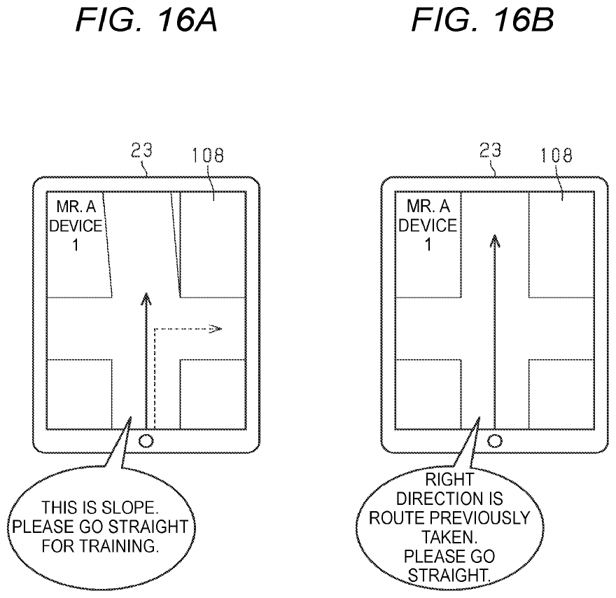

In this case, as shown in FIG. 16A, the display panel 108 of the tablet terminal 23 displays a display content including an image for identifying the user (“Mr./Ms. A”) and the individual walking assistant vehicle 21 (“Device 1”), an image for identifying the optimal route, and an image for urging the user to take the optimal route. In addition, as such an image for urging the user to take the optimal route, for example, a message such as “This is a slope. Please go straight for training.” is displayed on the display panel 108 of the tablet terminal 23. In the present embodiment, the display panel 108 of the tablet terminal 23 that notifies the user by displaying an image for urging the user to take the optimal route corresponds to the walking environment providing function unit. In the present embodiment, the tablet control unit 109 generates the route information so as to be able to be used in the process for notifying with the display panel 108 of the tablet terminal 23, thereby providing the route information to the display panel 108.

The operation of the present embodiment will be described.

According to the present embodiment, when the user goes out using the walking support equipment 20, the tablet control unit 109 in the tablet terminal 23 executes the process of step S83 and the process of step S85 in order to improve the efficiency of improving the physical condition by walking using the walking support equipment 20. In the process of step S83, the optimal route in which the second routes that improve the user's PCI are extracted among the routes from the current location to the destination and the third route that makes the user feel fresh when going out is preferentially extracted among the second routes is notified. As a result, the user can be urged to take the optimal route. Further, in the process of step S85, the optimal route in which the second routes that improve the user's PCI is extracted from the routes starting from the current location until returning to the current location and the third route that makes the user feel fresh is preferentially extracted from the second routes is notified. As a result, the user can be urged to take the route.

The effects of the present embodiment will be described.

(5) According to the present embodiment, the user can go out using the walking support equipment 20 in an environment where the efficiency of improving the physical condition is improved, which can contribute to the construction of a system that provides a good walking environment to the user who is out.

(6) When notifying the user of the second route, if the same route is notified each time, it is conceivable that the user may feel tired of going out using the walking support equipment 20. In this case, there is a concern that the user's motivation to go out using the walking support equipment 20 will decrease and the efficiency of improving the physical condition will decrease. In this regard, according to the present embodiment, by preferentially notifying the user of the third route different from the walking history of the user by the display panel 108, which is the walking environment providing function unit, it is possible to devise ways to make the user feel fresh when going out using the walking support equipment 20.

(7) Route information indicating the optimal route suitable for improving the physical condition is generated and prepared on the system in default. In this case, there is a request from the user to notify the optimal route suitable for improving the physical condition, the route information generated and prepared can be used to immediately respond to the user's request.

In addition, each of the above-mentioned embodiments may be changed as follows. In addition, the other embodiments below can be combined with each other to the extent that there is no technical inconsistency.

In the third embodiment, when the tablet control unit 109 generates the route information indicating the optimal route, it is sufficient that either the second route or the third route can be extracted as the optimal route.

In the third embodiment, the function of inputting destination information can be deleted. In this case, among the processes executed by the tablet control unit 109, the process of step S81, which is the process of collecting destination information, may be deleted. In this case, the tablet control unit 109 may randomly extract a route from the current location, or may extract the route among the routes starting from the current location and returning to the current location in the same manner as in step S83.

In the third embodiment, the optimal route may be extracted only when the destination information is input, that is, when step S81: YES.

In the second embodiment, the tablet control unit 109 only needs to have any of the function that notifies the first route among the routes from the current location to the destination so as to avoid the caution places, that is, the process of step S73, based on the caution place map, and the function that notifies that the user is heading to a caution place, that is, the processes of steps S75 and S76.

In the second embodiment, the area to which the once acquired caution place map is applied may be updated while going out using the walking support equipment 20. In this case, after the tablet control unit 109 once acquires the caution place map, for example, when the location information is likely to deviate from the area to which the caution place map is applied, the tablet control unit 109 may reacquire a caution place map around the point indicated by the location information. Further, the tablet control unit 109 may acquire and update the caution place map at predetermined time intervals while going out. This also applies to the third embodiment.

In each of the above embodiments, the environmental information may be an entertainment place map which is information showing so that a suitable place (hereinafter, referred to as “entertainment place”) for giving enjoyment when walking can be grasped. Regarding the location indicated by the location information, such an entertainment place is set based on the location status information, which is information indicating, for example, a famous place, a landscape, a store, an entertainment facility, a construction site, date and time, an event, and the like. In this case, in the server 30, the entertainment place map is stored in a predetermined area of the storage unit 113. Further, the tablet control unit 109 generates route information indicating a route based on the entertainment place map acquired through the server 30.

As an optimal route that can motivate the user to go out using the walking support equipment 20, for example, a route that can enhance the walking comfort of the user while going out by the walking support equipment 20, or a route that can entertain the user while going out by the walking support equipment 20 can be considered. That is, in addition to the above-described embodiments, an embodiment that realizes the above-mentioned function of notifying a route that can enhance the comfort of the user or entertain the user is effective from the viewpoint of motivating the user to go out using the walking support equipment 20.

In the third embodiment, the load place may be set in the location indicated by the location information based on, for example, the weather such as the wind direction and the air volume at the location, the climate, and the like.

In the third embodiment, in the processes of steps S83 and S85 of FIG. 15, the tablet control unit 109 can use information that can be acquired from the walking support equipment 20, such as information about the stride length in addition to the walking distance and the walking speed, the information about the arm swing, and continuous walking time, instead of PCI.

In the second embodiment, when extracting the first route, a function of adding conditions such as the shortest distance and the required time may be added. In this case, the tablet control unit 109 may extract a plurality of routes based on the above conditions and select a route that avoids the caution places from the extracted routes. If the tablet control unit 109 does not have a route to avoid the caution places, the tablet control unit 109 may notify the route considered to be the safest among the extracted routes as the first route.

In the first embodiment, the outing area was specified based on the walking history but the user may be able to set the outing area by himself/herself or a person concerned with the user may be able to set the outing area.

In the second embodiment, as the caution place map, the user may be able to set the caution places that he/she wants to avoid, or a person concerned with the user may be able to set the caution places that he/she wants to avoid. This also applies to the third embodiment.

In the second and third embodiments, the method of detecting unevenness, slopes, the presence or absence of a groove, and the like is not limited to using the detection result of the acceleration sensor G and, for example, the detection result of the vibration sensor or the result of image analysis of camera images can also be used.

In each of the above embodiments, the tablet control unit 109 of the tablet terminal 23 executes the processes of steps S52 and S53 shown in FIG. 8, steps S73, S75 and S76 shown in FIG. 12, and steps S83 and S85 shown in FIG. 15 as the process execution step. However, these processes may be executed by the server 30 or the facility terminal 40. In this case, in the first embodiment, the server 30 and the facility terminal 40 may be configured to execute the processes corresponding to steps S17 and S51 to S53 to execute the location information collection step, the environmental information collection step, and the process execution step. On the other hand, the tablet control unit 109 may be configured to execute the process execution step based on whether or not the wandering information is received from the server 30. Further, in this modification, the tablet terminal 23 that notifies the user's wandering through an image display corresponds to the walking environment providing function unit. This also applies to the second and third embodiments. In the first embodiment, the family terminal 50 may be configured to execute the process execution step instead of the tablet terminal 23 and the server 30.

In the second embodiment, when the destination information is input and the destination is set, the tablet control unit 109 may first generate the route information of the first route from the location indicated by the location information at that time to the destination. After that, the tablet control unit 109 may execute the notification of the route based on the first route generated first. This also applies to the third embodiment.

In each of the above embodiments, by storing the environmental information in the storage unit of the tablet terminal 23, various information may not be acquired from the server 30 while going out using the walking support equipment 20. In this case, the tablet control unit 109 may execute a process for providing a walking environment based on the environmental information stored in the storage unit. In this modification, as the tablet terminal 23, a tablet terminal that does not have a function of transmitting and receiving various information through the network can be used.

In each of the above-described embodiments, the configuration may be made to have a function of generating information necessary for realizing the functions of the above-mentioned embodiments, such as wandering information, route information, and warning information, regardless of the purpose. That is, there is information that is generated and used according to the function of the walking support system 10 and information that is generated but not used. In this case, regardless of the function of the walking support system 10, for example, the programs stored in the memory can be shared and the versatility of the memory can be enhanced.

In each of the above embodiments, the map data used when generating the environmental information is not limited to the one generated in steps S41 to S43, and may be map data including only the basic information created by the map provider, or the like and can be changed as appropriate.

In each of the above embodiments, when logging in to the walking support system 10, instead of inputting login information to the tablet terminal 23, a face recognition system that authenticates the user's face to be used as login information, or fingerprint recognition system that authenticates a user's fingerprint to be used as login information may be adopted.

In each of the above embodiments, the location information sensor 120 may be provided on the walking assistant vehicle 21 or the wearable terminal 22 and only needs to be provided in the walking support equipment 20. Further, the acceleration sensor G may be provided on the wearable terminal 22 or the tablet terminal 23 and only needs to be provided in the walking support equipment 20.

In each of the above embodiments, the location information sensor 120 is not limited to the GPS sensor and the current location may be estimated from the rotation speeds of the front wheels 91 and the rear wheels 92 from the place where the walking support device 20 is started. In this case, the location information sensor 120 uses, for example, the location of the support facility having the walking support device 20 as the place where the walking support device 20 is started.

In each of the above embodiments, the walking support equipment 20 may remove the wearable terminal 22 from the configuration so that the walking assistant vehicle 21 includes the heart rate sensor 106. Further, the walking support equipment 20 may remove the tablet terminal 23 from the configuration so that the walking assistant vehicle 21 has the function of the tablet terminal 23. In this case, the walking support equipment 20 may remove the wearable terminal 22 from the configuration and include only the walking assistant vehicle 21.

In each of the above embodiments, the walking assistant vehicle 21, the wearable terminal 22, and the tablet terminal 23 may transmit and receive various information through Wi-Fi (registered trademark), that is, a wireless LAN of any standard such as IEEE802.11a or IEEE802.11g. In this case, the tablet terminal 23 is connected to the network via a wireless LAN provided in the support facility or is connected to the network via a wireless LAN provided in the smartphone owned by the user.

In each of the above embodiments, the content to be displayed as an image on the tablet terminal 23, the facility terminal 40, and the family terminal 50 may be appropriately changed as long as the content can reflect the intention to be expressed. For example, in the first embodiment, as shown in FIG. 9C, when the facility terminal 40 or the family terminal 50 receives the wandering information, a display content including a map image for identifying the current location of the user may be displayed on the display panel 40a, which is the display screen of the facility terminal 40, or the display panel 50a, which is the display screen of the family terminal 50. Further, as shown in FIG. 16B, the display panel 108 of the tablet terminal 23 may display, for example, a message image such as “The right direction is the route previously taken. Please go straight.” as an image for urging the user to take the optimal route.

In the first embodiment, the tablet terminal 23 may output the sound of the content notifying the wandering instead of displaying the image of the content notifying the wandering on the display panel 108. This also applies to the second and third embodiments.

In each of the above embodiments, the server 30 is a computer configured one or more processors that execute computer programs, or one or more dedicated hardware circuits such as integrated circuits for specific applications that execute at least a part of various processes, or a circuit including a combination of the processor and the dedicated hardware circuit. The computer includes a CPU and memory. RAM, ROM, or the like is adopted as the memory. The memory stores a program code or a command configured to cause the CPU to execute the process. The memory, in other words, the computer-readable medium may be configured by any available medium accessible by a general purpose or dedicated computer. The configuration of the computer is the same for the facility terminal 40 and the family terminal 50, in addition to the walking assistant vehicle control unit 95, the wearable terminal 22, and the tablet terminal 23 of the walking assistant vehicle 2 L

What is claimed is:

1. A system for providing a walking environment comprising:
a controller,
wherein the controller is configured to execute as various processes:
a location information collection step that collects a location information of a walking support equipment that supports walking;
an environmental information collection step that collects an environmental information used upon providing the walking environment while going out; and
a process execution step that executes a process of providing the walking environment to a user that walks with the walking support equipment, based on the environmental information collected in the environmental information collection step and the location information collected in the location information collection step.

2. The system for providing the walking environment according to claim 1, further comprising:
a network communicator configured to receive the environmental information collected in the environmental information collection step by a communicating via a network.

3. The system for providing the walking environment according to claim 1, wherein when the user is wandering, the controller notifies the user or a person concerned with the user of that the user is wandering, by the walking environment provided to the user by the process executed in the process execution step.

4. The system for providing the walking environment according to claim 3, further comprising:
a walking environment providing function unit configured to notify the user or a person concerned with the user of that the user is wandering,
wherein the environmental information collection step includes collecting an outing area as the environmental information, the outing area being information indicating an area where the user while going out can walk, and
wherein the process execution step includes an information providing step of providing information indicating that the user is wandering to the walking environment providing function unit when the location information deviates from the outing area.

5. The system for providing the walking environment according to claim 1, wherein the controller notifies the user of a route suitable for ensuring a walking safety upon using the walking support equipment, by the walking environment provided to the user in the process execution step.

6. The system for providing the walking environment according to claim 5, further comprising:
a walking environment providing function unit configured to notify the user of that the user is heading to a place unsuitable for ensuring the walking safety,
wherein the environmental information collection step includes collecting information indicating the place unsuitable for ensuring the walking safety upon using the walking support equipment as the environmental information, and
wherein the process execution step includes an information providing step of providing information indicating that the user is heading to the place unsuitable for ensuring the walking safety to the walking environment providing function unit when the location information is changed such that the walking support equipment is heading to the place unsuitable for ensuring the walking safety.

7. The system for providing the walking environment according to claim 5, further comprising:
a walking environment providing function unit configured to notify the user of a first route among routes to a destination of the user so as to avoid the place unsuitable for ensuring the walking safety upon using the walking support equipment,
wherein the controller is configured to further execute a destination information collection step that collects a destination information, the destination information is information indicating the destination of the user,
wherein the environmental information collection step includes collecting information indicating the place unsuitable for ensuring the walking safety upon using the walking support equipment as the environmental information, and
wherein the process execution step includes an information providing step of providing information on the first route to the walking environment providing function unit.

8. The system for providing the walking environment according to claim 5, wherein the process execution step includes generating a route information, the route information being information indicating the route suitable for ensuring the walking safety upon using the walking support equipment, and being information configured to notify the user of the route.

9. The system for providing the walking environment according to claim 1, wherein the controller notifies the user of a route suitable for improving a physical condition of the user, by the walking environment provided to the user in the process execution step.

10. The system for providing the walking environment according to claim 9, further comprising:
a walking environment providing function unit configured to notify the user of a second route suitable for increasing efficiency of improving the physical condition of the user, based on a walking ability of the user,
wherein the controller is configured to further execute a walking ability information collection step that collects a walking ability information indicating the walking ability of the user,
wherein the environmental information collection step includes collecting a load information when the user walks with the walking support equipment at each place as the environmental information, and
wherein the process execution step includes an information providing step of providing information on the second route to the walking environment providing function unit.

11. The system for providing the walking environment according to claim 10, wherein the walking ability information collection step includes collecting a physiological cost index as the walking ability information, the physiological cost index indicating a load during walking as an index based on a heart rate of the user.

12. The system for providing the walking environment according to claim 9, further comprising:
a walking environment providing function unit configured to notify the user preferentially of a third route that is different from a history of a walking route while the user is out,
wherein the environmental information collection step includes collecting information indicating the history of the walking route of the user while going out as the environmental information, and
wherein the process execution step includes an information providing step of preferentially providing information on the third route to the walking environment providing function unit.

13. The system for providing the walking environment according to claim 9, wherein the process execution step includes generating a route information, the route information being information indicating the route suitable for improving the physical condition of the user, and being information configured to notify the user of the route.

14. A system for supporting walking comprising:
a server system including a server controller that executes various processes, a server storage unit that stores various information, and a server network communicator that transmits and receives the various information by communicating via a network; and
a walking support equipment including a walking support equipment controller that executes various information and a walking support equipment network communicator that transmits and receives the various information by communicating via the network,
wherein the walking support equipment controller builds a system for providing a walking environment configured to execute:
a location information collection step that collects a location information of the walking support equipment;
an environmental information collection step that collects an environmental information used upon supporting the walking environment while going out by the communicating of the walking support equipment network communicator; and
a process execution step that executes a process of providing the walking environment to a user of the walking support equipment, based on the environmental information collected in the environmental information collection step and the location information collected in the location information collection step.

15. A method for providing a walking environment comprising:
a location information collection step that collects a location information of a walking support equipment;
an environmental information collection step that collects an environmental information used upon providing the walking environment while going out; and
a process execution step that executes a process of providing the walking environment to the user, based on the environmental information collected in the environmental information collection step and the location information collected in the location information collection step.

16. A non-transitory computer readable medium storing a program causing a computer to execute the method for providing the walking environment according to claim 15.
